(19) United States
(12) Patent Application Publication
(10) Pub. No.: US 2024/0108272 A1
(43) Pub. Date: Apr. 4, 2024

(54) EEG ELECTRODE ARRAY AND METHOD OF USE

(71) Applicants: ICHILOV TECH LTD., Tel Aviv (IL); RAMOT AT TEL AVIV UNIVERSITY LTD., Tel Aviv (IL)

(72) Inventors: Talma HENDLER, Tel Aviv (IL); Mordekhay MEDVEDOVSKY, Beer Sheva (IL); Tomer GAZIT, Tel Aviv (IL); Evgeny TSIZIN-GOLDMAN, Tel Aviv (IL); Alexander BRONSTEIN, Tel Aviv (IL)

(73) Assignees: ICHILOV TECH LTD., Tel Aviv (IL); RAMOT AT TEL AVIV UNIVERSITY LTD., Tel Aviv (IL)

(21) Appl. No.: 18/557,965

(22) PCT Filed: Mar. 11, 2021
(86) PCT No.: PCT/IL2021/050270

§ 371 Date: Oct. 30, 2023

Related U.S. Application Data
(60) Provisional application No. 62/988,984, filed on Mar. 13, 2020.

(51) Int. Cl. A61B 5/372; A61B 5/00; A61B 5/256; A61B 5/271; A61B 5/291

(52) U.S. Cl. CPC A61B 5/372; A61B 5/256; A61B 5/271; A61B 5/291; A61B 5/4094; A61B 5/7203; A61B 2562/046

(57) ABSTRACT

Provided herein are methods for EEG monitoring of a patient, which includes: positioning an array comprising a plurality of EEG electrodes on the patient's head; recording, during a first time period, a first data set of EEG signals from a first set of spatial scalp locations; manipulating the array to record, during a second time period, a second data set of EEG signals from a second set of spatial locations including at least some spatial scalp locations that are different from the first set of spatial scalp locations; integrating the first and second data sets, thereby generating a combined data set with a higher spatial density; and processing the combined data set to determine EEG events.

### FIELD OF THE INVENTION

The present invention, in some embodiments thereof, relates to a device and method for recording EEG signals, and, more particularly, but not exclusively, to EEG monitoring in which only a selected partial set of electrode positions are used for recording at a given time.

### BACKGROUND OF THE INVENTION

Electroencephalography (EEG) is used to monitor or record spontaneous electrical activity of the brain over a period of time. EEG usually uses electrodes which are placed along the scalp, non-invasively. EEG measures voltage fluctuations resulting from current within the neurons of the brain.

EEG can be used to diagnose various conditions, such as epilepsy, sleep disorders, depth of anesthesia, coma, encephalopathies, brain death, and the like as well as focal brain conditions, such as, tumors and stroke.

A publication by some of the inventors of this application is titled “Virtual MEG Helmet: Computer Simulation of an Approach to Neuromagnetic Field Sampling”, IEEE Journal of Biomedical and Health Informatics, Vol. 20, No. 2, March 2016 539. A publication by G. Lantza, R. Grave de Peraltaa, L. Spinellia, M. Seeckc, and C. M. Michela, is entitled: “Epileptic source localization with high density EEG: how many electrodes are needed?” (Clinical Neurophysiology 114 (2003) 63-69).

Nevertheless, there is a need in the art for improved systems and methods for EEG monitoring of subjects, which exhibit increased spatial density and which are accurate, sensitive, cost and time efficient.

### SUMMARY OF THE INVENTION

According to an aspect of some embodiments there is provided a method for EEG monitoring of a subject, the method includes: positioning an array comprising a plurality of EEG electrodes on the patient's head; recording, during a first time period, a first data set of EEG signals from a first set of spatial scalp locations; manipulating the array to record, during a second time period, a second data set of EEG signals from a second set of spatial locations including at least some spatial scalp locations that are different from the first set of spatial scalp locations; integrating the first and second data sets, thereby generating a combined data set with a higher spatial density; and processing the combined data set to determine EEG events.

In some embodiments, the first data set is recorded by a first set of electrodes of the array and the second data set is recorded by a second set of electrodes of the array.

In some embodiments, manipulating comprises moving the second set of electrodes from a position in which the electrodes are away from the scalp to a position in which the electrodes contact the scalp.

In some embodiments, at least some locations of the first set of spatial scalp locations and of the second set of spatial scalp locations overlap.

In some embodiments, recording is carried out by placing the first set of electrodes or the second set of electrodes in pressurized contact against the scalp.

In some embodiments, each of the first set of electrodes and the second set of electrodes include less than the total number of electrodes of the array.

In some embodiments, a duration of each of the first and second time periods is shorter than 30 minutes.

In some embodiments, the method comprises repeating recording from the first and second sets of spatial scalp locations in an alternating manner over a total duration of between 1-3 days or longer.

In some embodiments, processing comprises determining EEG events associated with epileptic seizures.

In some embodiments, integrating comprises representing data recorded by each electrode using a 4-dimensional vector which includes coordinates of the electrode position and a voltage amplitude recorded by the electrode.

In some embodiments, generating the combined data set comprises determining low spatial frequency components of EEG events from each of the first and second data sets and clustering the low spatial frequency components according to their temporo-spatial characteristics.

In some embodiments, the method comprises revealing high spatial frequency components of EEG events from the combined data set.

In some embodiments, the method comprises assessing current electrode positions using photogrammetry methods.

According to an aspect of some embodiments there is provided a system for EEG monitoring of a patient, which includes: a cap shaped and sized for fitting onto the head of the patient; the cap includes: a plurality of dry EEG electrodes including at least a first set of electrodes and a second set of electrodes; an actuator configured to press the first set of electrodes against the patient's scalp to record EEG signals from a first set of spatial scalp locations, and then to press the second set of electrodes against the patient's scalp to record EEG signals from a second set of spatial scalp locations at least partially different than the first set of spatial scalp locations; and
a processor programmed to interpolate data recorded by the first set of electrodes and the second set of electrodes to determine EEG events.

In some embodiments, the actuator is activated by a switch or timer.

In some embodiments, when the actuator is activated, the actuator presses the first set of electrodes or the second set of electrodes against the scalp for a time period shorter than 30 minutes.

In some embodiments, the electrodes are mounted onto inflatable tubes defined within the cap and wherein the actuator is configured to inflate the tubes to move the electrodes into contact with the scalp.

In some embodiments, each electrode is operably attached to a spring, wherein deflation of a tube releases tension on the spring, causing the spring to bounce distally, thereby retracting the electrode away from the scalp.

In some embodiments, the first set of electrodes is mounted onto a first tube or set of tubes, and wherein the second set of electrodes is mounted onto a second tube or set of tubes, the first and second tube or sets of tubes independently inflatable.

In some embodiments, the electrodes are mounted or embedded within compression bands which extend at least across a long and transverse axis of the cap, the bands configured to be pulled on to push the electrodes against the scalp.

In some embodiments, the cap comprises fixtures configured for attachment onto designated anatomical landmarks on the patient's head.

In some embodiments, the landmarks comprise two or more of: the nasion, the inion and the preauricular point.

In some embodiments, a size of the cap is adjustable via one or more pullable straps for obtaining a personal fit to the patient's scalp.

In some embodiments, the system comprise a user interface in communication with the processor, the user interface enabling self-activation by the patient or by a caregiver.

In some embodiments, the processor is in communication with a remote server for transferring the recorded data and/or for comparing the recorded data onto data stored on the remote server.

In some embodiments, the switch or timer are configured to activate the actuator to alternate between the first and second electrode sets throughout a total duration of between 1-5 days.

In some embodiments, the cap is constructed such that each of the first and second sets of electrodes, when positioned in contact with the scalp, are distributed such between 2-8 electrodes are placed on each of the forelock, midscalp and crown regions of the scalp.

According to an aspect of some embodiments there is provided a system for EEG monitoring of a patient, comprising: an array comprising a plurality of EEG electrodes; an actuator configured for moving two or more of the plurality of electrodes together from a first position in which the electrodes are away from the patient's scalp to a second position in which the electrodes are in operable contact with patient's scalp.

In some embodiments, the actuator is configured to maintain, at any given time during monitoring, at least some electrodes out of the plurality of electrodes at the first position away from the patient's scalp.

According to an aspect of some embodiments there is provided a system for EEG monitoring of a patient, comprising: a cap shaped and sized for fitting onto the patient's scalp; the cap includes: a plurality of EEG electrodes embedded within it; the cap moveable from a first orientation relative to the scalp in which the plurality of electrodes are positioned to record EEG signals from a first set of spatial locations to a second orientation relative to the scalp in which the plurality of electrodes are positioned to record EEG signals from a second set of spatial locations; and a processor programmed to integrate recordings of the first and second orientations to determine EEG events.

In some embodiments, a total number of electrodes is smaller than the total number of spatial locations being recorded.

In some embodiments, the total number of electrodes is half the number of spatial locations being recorded.

According to an aspect of some embodiments there is provided a method for long term EEG monitoring of a patient, comprising: for a time period of between 1 hour −7 days, monitoring EEG of a patient by recording signals from different sets of spatial scalp locations; and integrating data recorded from the different sets of spatial locations, wherein integrating increases a spatial resolution of the data so that EEG events are detected as if the events were recorded from both of the sets of spatial locations.

In some embodiments, integrating increases the spatial resolution by between 20-200%.

According to an aspect of some embodiments there is provided an integrated EEG electrode and glue pad comprising: a flexible glue pad onto which an EEG electrode sensor is centrally mounted, the flexible glue pad surrounded by frame which is rigid enough to prevent portions of the glue pad from sticking to each other.

According to an aspect of some embodiments there is provided a method for reducing noise from an EEG recording, comprising: prior to recording EEG from a patient's scalp, short circuiting EEG electrodes by pairing the electrodes together; assessing an artifact sensed by the short-circuited electrodes; recording EEG from the patient's scalp using a different set of electrodes while maintaining the paired electrodes away from the scalp; removing the artifact from the EEG data recorded by the different set of electrodes.

According to an aspect of some embodiments there is provided a method for long term EEG monitoring of a patient, the method includes: applying electrodes to the patient's scalp by positioning a first set of electrodes at a first distribution along a scalp region of interest; and a second set of electrodes at a second distribution along the other scalp regions; wherein electrode density of the first distribution is at least 60% higher than electrode density of the second distribution.

In some embodiments, the first set of electrodes and the second set of electrodes are placed in different/discrete time points.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a general method for EEG monitoring, according to some embodiments;

FIGS. 2A-B are schematic illustrations of systems configured for variable positioning of electrodes on the patient's scalp, according to some embodiments;

FIG. 3 schematically illustrates electrode positions at selected spatial scalp locations during different recording sessions, according to some embodiments;

FIG. 4 is a flowchart of a method for EEG monitoring by creating a virtual dense array of EEG electrodes, according to some embodiments;

FIGS. 5A-B are a flowchart of a method for processing EEG signals obtained by two different sparse electrode arrays at two different time instances (5A) and a detailed flowchart of interpolation of data recorded at the two different time instances (5B), according to some embodiments;

FIG. 6 is a flowchart of a method for EEG monitoring in which electrodes are placed in contact with the scalp for a limited time period, according to some embodiments;

FIG. 7 is a schematic illustration of a cap comprising a mechanism for positioning selected electrodes against the scalp, according to some embodiments;

FIGS. 8A-B are schematic illustrations of a cap comprising hydraulic or pneumatic tubes for positioning electrodes against the scalp (8A) and a schematic drawing of a single electrode being pressured against the scalp (8B), according to some embodiments;

FIG. 9 is a schematic illustration of an integrated electrode and conductive glue pad, according to some embodiments;

FIGS. 10A-C illustrate a method for measuring and marking electrode positions on the patient's scalp, according to some embodiments;

FIG. 11 is a flowchart of a method of reducing noise from an EEG recording, according to some embodiments;

FIG. 12 schematically illustrates pairing of electrodes in a cap comprising dry electrodes, according to some embodiments;

FIG. 13 schematically illustrates pairing of electrodes in a cap comprising wet electrodes, according to some embodiments;

FIG. 14 is a schematic illustration of a cap comprising a spring-actuated mechanism for mounting and/or dismounting the EEG electrodes on the scalp, according to some embodiments;

FIGS. 15A-C are graphical examples of an electric field recorded by a 64 channel EEG array (FIG. 15C) and electric fields constructed from data of two subsets of electrode arrays, each including 32 electrodes (FIG. 15A, FIG. 15B), according to some embodiments;

FIG. 16 is an example of a sparsely distributed electrode array on the scalp, according to some embodiments;

FIG. 17 illustrates a simulation performed in accordance with some embodiments, simulating EEG recording at 4 different time instances using a sparse electrode set of 16 electrodes, and interpolating the recordings to obtain a measurement equivalent to one performed by use of 64 electrodes simultaneously;

FIG. 18A shows a pictogram of a first cap (Cap 1), placed on a subject head, with electrodes labeled by numbers;

FIG. 18B shows a pictogram of a second cap (Cap 2) placed on a subjected head, with labeled (black numbers) electrodes and further shows the corresponding places of the cap 1-electrodes, which are labeled by black circles and corresponding (red) numbers;

FIG. 19A shows EEG trace referenced to the electrode placed in T2 position, as measured by the first cap (Cap 1);

FIG. 19B shows EEG trace referenced to average of all electrodes on the first cap (Cap 1);

FIG. 20A shows EEG trace referenced to the electrode placed in T2 position, as measured by the second cap (Cap 2);

FIG. 20B shows EEG trace referenced to average of all electrodes on the second cap (Cap 2);

FIG. 21 shows line graph of one real part of FFT calculated for 10 Hz bin of cap 1 data. X-axis—channel numbers. Y-axis Real part of signal power in 10 Hz in 1 sec;

FIG. 22 shows line graphs of 11 real parts of rotated FFT for cap 2 data (each with 0.1 pi radians rotated step). X-axis—channel numbers. Y-axis Real part of signal power in 10 Hz in 1 sec;

FIG. 23—line graphs of the calculated field values: Graph line 1800 (Yellow)—shows the calculated field values (of 10-Hz bin) in the interpolated points between cap 1 electrodes. Graph line 1802 (Green) shown the field values at cap 2 electrodes corresponding to cap 1 interpolated points;

FIG. 24 shows reconstructed field without amplitude normalization. Graph line 1810 (Blue)—cap 1. Graph line 1812 (Red)—cap 2. X-axis—channel numbers. Y-axis—real part of signal power in 10 Hz in 1 sec; and

FIG. 25 shows a reconstructed field. Graph line 1820 (Blue)—cap 1. Graph line 1822 (Red)—cap 2. X-axis—channel numbers. Y-axis—real part of signal power in 10 Hz in 1 sec (for cap 2—normalized according to cap 1).

### DETAILED DESCRIPTION

The present invention, in some embodiments thereof, relates to a device and method for recording EEG signals, and, more particularly, but not exclusively, to EEG monitoring in which only a selected partial set of electrode positions are used for recording at a given time. Methods and systems for example as described herein may be especially advantageous for long term EEG monitoring at home, for example for the purpose of detecting and observing seizures of a patient suffering from epilepsy.

An aspect of some embodiments relates to long term EEG monitoring in which EEG is recorded by at least two sets of sparse electrode positions in an alternating manner, such that at a given time during monitoring only one of the sets actively records EEG from the patient's scalp. In some embodiments, data recorded by the two sets is processed and integrated, generating a data set with increased spatial density of electrodes positions. In some embodiments, EEG events are detected from the combined data as if these events were recorded by a denser set of electrode positions, including both of the different sparse sets of electrode positions.

In some embodiments, processing and integrating of the recorded data involves clustering the data according to low spatial frequency components of the signal (which are commonly associated with deep brain activity) to reveal high spatial frequency components of the data (commonly associated with more superficial brain activity, detectable via a denser electrode array).

An aspect of some embodiments relates to a system for long term EEG monitoring designed for home usage, in which only a partial set of sparsely distributed electrodes actively records EEG from the patient at any given time during monitoring. In some embodiments, the system comprises an electrode array including an actuator configured for one or both of moving the array with respect to the patient's head so as to reposition electrodes on the scalp, and/or for moving electrodes from a position in which the electrodes are located away from the scalp to a position in which the electrodes are operably attached to the scalp. In an example, the actuator is configured to lift a first set of electrodes away from the scalp and advance a second set of electrodes into contact with the scalp. The second set of electrodes may record for a defined time session, and then the sets are switched so that the first set is brought into contact with the scalp for measuring for a second defined time session. Optionally, some of the spatial scalp locations overlap between the sessions.

Some potential advantages of limiting a time period in which a certain set of spatial scalp locations are being recorded from may include: reducing risks involved with prolonged applying of pressure onto the scalp (for example if dry electrodes are used); reducing pain and discomfort to the user.

In an exemplary construction, the electrode array defines a cap shape or a frame having a geometry suitable for fitting on the patient's head (e.g., rounded, egg-shaped, concaved). In some embodiments, the cap comprises inflatable tubes or chambers which are positioned to move, upon inflation (with fluid and/or air), a selected set of electrodes into contact with the scalp. Optionally, upon deflation, the electrodes are lifted away from the scalp. Additionally, or alternatively, electrodes are mounted or embedded within compression bands which extend at least across a long and transverse axis of the cap, the bands configured to be tensioned to push the electrodes against the scalp or released to lift the electrodes away from the scalp. In some embodiments, electrode movement is actuated using an elastic element, for example a spring. In an example, an electrode is operably coupled to a spring, and by releasing tension of the spring the spring may bounce back to retract the electrode away from the scalp.

In some embodiments, switching between the sets of electrode positions is controlled by a controller, optionally via a switch or timer element. In some embodiments, predefined time periods are modified (shortened or lengthened) with respect to a number of EEG events (e.g., epileptic seizures) identified during the recording. Optionally, one or more electrode positions are modified in real time.

In some embodiments, different sets of electrodes may each be placed on a separate cap, that may be interchangeably be worn by the subject, to obtain tow discrete measurements for various spatial and/or temporal locations, according to the location/position/distribution of the electrodes in a set.

In some embodiments, electrodes are distributed in a homogenous distribution across the scalp. In some embodiments, the head is divided into 2, 3, 4, 5, 6, 8 or intermediate, larger or smaller number of regions, and electrodes are distributed such that at least 1, 3, 5, 7, 10 electrodes or intermediate, larger or smaller number are positioned to record from each region. In some embodiments, for example when wet electrodes are used, using a relatively low number of electrodes may save time (due to that less electrodes need to be applied and adhered to the scalp) and may also reduce or prevent inter-electrode conductive bridges.

In some embodiments, during repetitive recording sessions (such as sessions recorded by the same electrode set), electrode positions are repeated so that measurement is performed from similar scalp locations. Alternatively, electrode positions are modified intentionally. Alternatively, an electrode position can be within some spatial limit relative to its position during one or more earlier recording sessions, for example within a radial distance of less than 5 mm away from a previous position, less than 3 mm away, less than 10 mm away or intermediate, longer or shorter distances, while still being close enough to record EEG signals which are compatible with the previous recordings. A potential advantage of allowing some freedom in electrode position may include saving time and effort which are needed for repeating an exact electrode position.

An aspect of some embodiments relates to reducing noise from an EEG recording by short-circuiting electrodes which are currently not being used for recording and using these electrodes as artifact sensors. In some embodiments, electrodes of a set that is not in contact with the scalp during a recording session are paired to be short circuited. An artifact sensed by the short-circuited electrodes can be removed from the recorded data, as the artifact is known. In some embodiments, EEG signals (recorded by electrodes that were not short circuited) which include a trace from a short-circuited electrode set, can later be removed from the data, for being correlated (in time) with an artifact.

It is noted that the term “cap” as referred to herein may encompass a housing or frame, a flexible net structure, a rigid or semi-rigid construction, and/or any other structure having a geometry which matches a human head and is configured to hold (or to have mounted onto) a plurality of electrodes or a pre-structured electrode array. In some embodiments, the cap is adjustable so as to obtain a personal fit with the patient's head anatomy. In some embodiments, the cap comprises one or more fixtures for coupling the cap to the patient's head and maintaining the cap in place. Optionally, the fixtures comprise clips or adhesives positioned to engage one or more anatomical landmarks such as the nasion, the inion and the preauricular point.

As used herein, the terms “patient” and “subject” may interchangeably be used.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

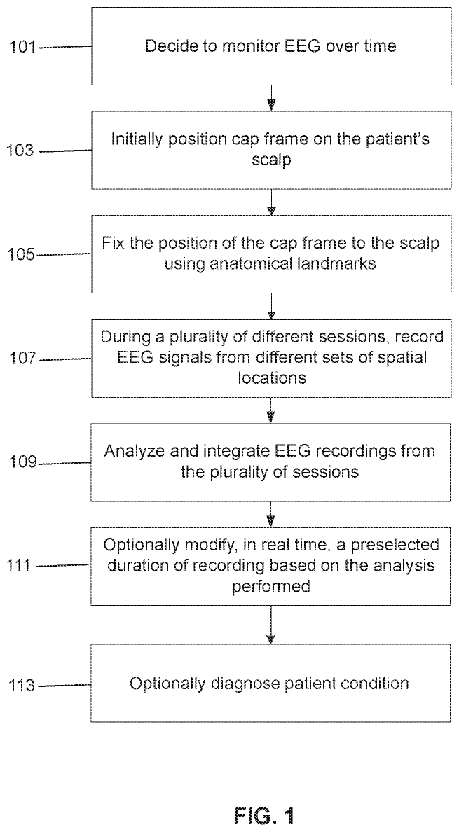

Referring now to the drawings, FIG. 1 is a flowchart of a general method for EEG monitoring, according to some embodiments.

EEG (Electroencephalography) is commonly performed for diagnosing conditions which affect the electrical activity of the brain, such as, but not limited to, epilepsy, sleep disorders, encalopathies, coma, brain death or damage and/or other conditions. A duration of EEG recording may range between several minutes, several hours, or, in some cases, several days. A method for example as described herein may be especially useful for long term monitoring (e.g., hours-days), but may also be applied for short periods of time.

In some embodiments, a decision to monitor EEG of a patient over time is made (101), for example by a physician, a neurologist and/or other clinical personnel. In some cases, in which a patient is diagnosed or suspected to suffer from epilepsy, long term monitoring is performed in order to record and optionally characterize one or more epileptic seizures. In some cases, the patient is admitted to the hospital and monitored, typically, for several days. In other cases, the patient is monitored at home, via ambulatory EEG.

In some cases, the patient is administered with antiepileptic drugs, which may affect a probability of a seizure occurring during monitoring.

In some embodiments, an EEG cap (and/or other suitable array arrangement of electrodes) is initially positioned on the patient's scalp (103) and fitted to the scalp according to anatomical landmarks (105). Optionally, the anatomical landmarks comprise one or more of the nasion, the inion and the preauricular point. In some embodiments, the cap comprises one or more attachment means for temporarily fixating the cap to the scalp, and the attachment means are located with respect to one or more of the anatomical landmarks.

In some embodiments, the process of initial fitting of the cap, taking into account a head anatomy and size of the specific patient, is performed at a hospital monitoring unit, clinic or lab. Optionally, initial fitting is carried out by a lab technician. Alternatively, the cap is configured to be adjustably fitted so that even initial setup can be carried out at home by the patient himself or by a caregiver. In some embodiments, initial fitting involves measurement of certain distances, for example a distance between electrodes and/or a distance between one or more electrodes and anatomical landmarks and/or a distance between anatomical landmarks, on the scalp, manually or digitally, for assessing electrode attachment locations. In some embodiments, photogrammetry methods are utilized for facilitating positioning of the electrodes on selected scalp locations. Optionally, electrode positioning is performed relative to the one or more anatomical landmarks. In some embodiments, misplacement of electrodes is compensated for by measuring the new (misplaced) electrode locations, and reassessing the electrode positions for the purpose of processing the recorded data. Optionally, photogrammetry methods are used for calculating the compensation for misplacement.

In some embodiments, during a plurality of different sessions, EEG signals are recorded from different sets of spatial locations (107). In some embodiments, during each session, only electrodes positioned at a selected set of spatial locations are used for recording. In some embodiments, at any given time throughout monitoring, only a partial set of electrodes out of a total number of electrodes of the cap are used for recording. Optionally, at a different recording session (for example a session performed successively), a different set of electrodes is used.

A duration of each session may range between, for example, 5 minutes-30 minutes, 10 minutes-1 hour, 1 hour-3 hours, 1 hour-1 day, or intermediate, longer or shorter time periods. A total monitoring duration (during which alternate sets of spatial locations are recorded from) may range between, for example, 15 minutes to 1 hour, 1 hour to 1 day, 2-4 days, or intermediate, longer or shorter time periods. In some embodiments, sessions are performed consecutively. Additionally or alternatively, sessions are performed with some overlap between them. Additionally or alternatively, sessions are performed with a recess period in between successive sessions.

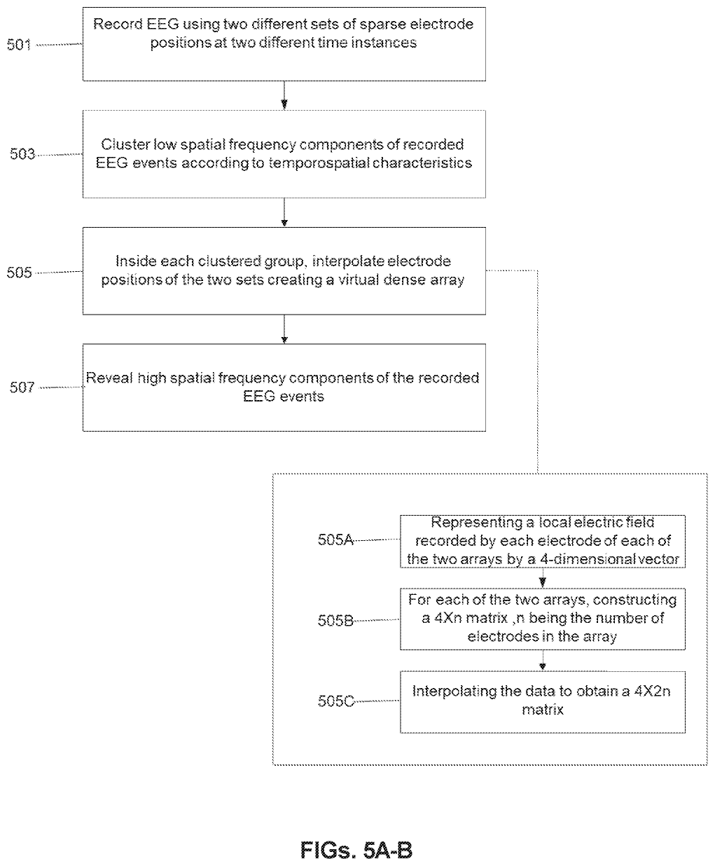

In some embodiments, EEG recordings from the plurality of sessions are analyzed and integrated (109). In some embodiments, analysis and integration of the recorded data comprises determining high frequency components of the signals by characterizing and comparing low frequency components of the signals. (see FIGS. 5A-B for an example of an analysis method).

In some embodiments, analysis comprises detection and optionally characterizing of neuro-electrographic events. Some examples of neuro-electrographic events include epileptic seizure, interictal epileptiform discharges.

In some embodiments, monitoring (e.g., recording in alternating sessions) is continued until a sufficient amount of data is collected. In an example, monitoring is continued until a sufficient number of EEG events that are required for diagnosing the patient are recorded. In a more specific example of a patient suffering from epilepsy, monitoring is continued until a sufficient number of seizures for determining a potential seizure onset area are recorded. Optionally, a preselected monitoring duration is modified in real time based on the analysis performed (111). For example, a recording duration may be shortened if a sufficient number of EEG events were observed, or instead lengthened it attempt to try and capture additional events.

In some cases, data obtained during the prolonged monitoring comprises ictalrecording (recording of a seizure) and inter-ictalrecording (recording in between seizures).

In some embodiments, EEG monitoring is accompanied by video observation. Optionally, the recorded EEG data and the video recording are observed simultaneously, and EEG events may be correlated with the observation of seizures or other behavior recorded on video. In some embodiments, for home monitoring, one or more video cameras are placed at the patient's home, for example in the patient's room and/or other rooms. Possibly, several cameras are used for obtaining several views of the patient. Additionally or alternatively, the EEG cap comprises an integrated video camera. Optionally, the camera is placed on a pullable extension of the cap, for example so that the patient can be recorded from some distance.

Optionally, a patient's condition is diagnosed based on the recorded data (113). In some embodiments, diagnosis is carried out by one or more of: transferring the data to a physician and/or a clinical center; comparing the data to data recorded from other patients or to the general population; comparing the data to known ranges or parameters (e.g. based on literature); comparing the data to previously recorded sessions of the same patient. In some cases, a preferred course of treatment is selected for the patient in accordance with the data obtained.

In some embodiments, the recorded data is integrated with and/or compared to data obtained by EEG-fMRI, MEG, and/or other relevant modalities. Optionally, for the purpose of integration and/or comparing, electrode positions relative to the scalp are maintained constant or are initially selected to match. In an example, initial fitting of a long term EEG cap for example as described herein may involve measuring and marking electrode positions according to a cap being used with a different modality, optionally, in a hospital setting.

In some embodiments, use of medications (such as for epilepsy treatment medications) during the monitoring period is prevented or limited, for example so that a likelihood of recording seizures is increased.

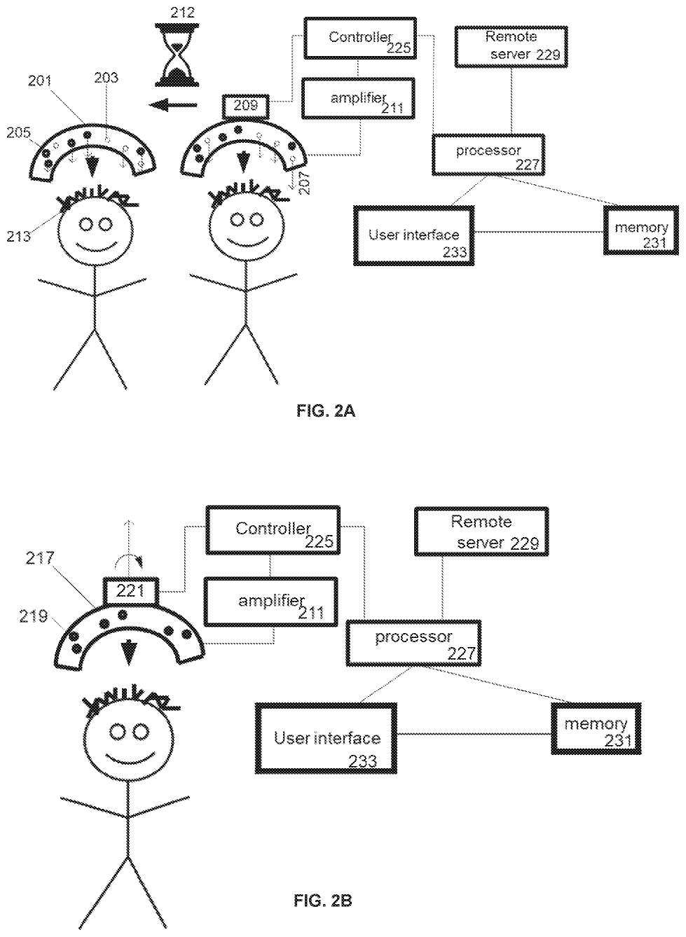

FIGS. 2A-B are schematic illustrations of systems configured for variable positioning of electrodes on the patient's scalp, according to some embodiments.

The systems schematically illustrated at FIGS. 2A, 2B are examples of EEG recording systems suitable for home monitoring. In some embodiments, the EEG cap (or other array arrangement of electrodes) is configured to be placed and optionally fitted to the patient's scalp by the patient themselves and/or by a caregiver (e.g., a family member). Optionally, the electrodes of the cap are attached to the cap itself (and/or to a holding frame) such that the electrode locations (relative to the other electrodes and/or to the cap) remain permanent. A potential advantage of a fixed arrangement of electrodes may include increasing a likelihood of placing the electrodes correctly on the patient's scalp, so that even after the cap is removed from the head and placed back on for additional monitoring, the electrodes are substantially placed on the same spatial scalp locations as they were before. In some embodiments, the cap comprises designated locations in which electrodes are positioned (e.g., cavities, markings, protrusions and/or other structures or indications for guiding electrode placement). In some embodiments, the cap includes or is connected to an amplifier to amplify voltage fluctuations recorded by the electrodes. The amplifier may be positioned on the patient body (e.g., as a wearable device), placed adjacent the patient, embedded within the cap, extending from the cap (so that it remains close to the patient head), and/or other positions.

FIG. 2A illustrates an example of a system comprising an EEG cap 201 which includes 2 electrode sets: 203 (indicated by the white circles) and 205 (indicated by the black circles). It is noted that other examples may include a plurality of different sets of electrodes, such as 3, 4, 5, 6 different electrode sets or intermediate, larger or smaller number. In some embodiments, each electrode set includes a plurality of electrodes positioned to be mounted onto a corresponding selected set of spatial scalp locations. In some embodiments, one or more electrodes of a first set may also be a part of the second set, so that one or more corresponding spatial scalp locations overlap and are monitored using both the first set and the second set. Data recorded by the electrodes is amplified by an amplifier and then digitized.

In some embodiments, cap 201 comprises a mechanism 209 for advancing a selected set of electrodes into operable contact with the scalp. In some embodiments, the mechanism is configured for moving (e.g., lifting) the set of electrodes away from the scalp. Such mechanism may include, but is not limited to, mechanical, hydraulic, pneumatic means for selectively positioning the electrodes. In some embodiments, electrode movement is limited only to an axial direction, for example, proximally along electrode axis 207 towards the scalp and/or distally along axis 207 away from the scalp. Optionally, electrodes that were lifted away from the scalp are received within designated pockets, cavities or channels formed in the cap.

In some embodiments, positioning (or re-positioning) of electrode sets is controlled using a timer 212 (for example implemented using a switch and/or other circuitry suitable for measuring time intervals). In an example, first electrode set 203 is actuated (e.g., moved) to be in operable contact with the patient's scalp 213 for a first predetermined time period, during which the electrical activity in the brain is recorded; at the end of the first predetermined time period, the second electrode set 205 is actuated (e.g. moved) to be in operable contact with the patient's scalp 213 for a second predetermined time period, during which the electrical activity in the brain is recorded. This process may be repeated during the full monitoring duration, alternating between the electrode sets.

In some embodiments, the first and second time periods partially overlap. In some embodiments, one or more electrodes of the first set are also used during recording from the second set or vice versa. Optionally, one or more spatial scalp locations are recorded from during the first time period and during a part of the second time period.

In some embodiments, the first and/or second time period are modified in real time. In some embodiments, a predetermined time period may be shortened if sufficient data was collected (for example if one or more seizures occurred during the monitoring); alternatively, a predetermined time period may be lengthened in order to collect more data.

FIG. 2B illustrates an example of a system comprising an EEG cap 217 comprising EEG electrodes 219. In some embodiments, the number of electrodes 219 is less than a full (dense) electrode array. In an example, cap 217 comprises 16. 32. 64, 128 or an intermediate, larger or smaller amount of electrodes. In some embodiments, cap 217 comprises less than 128 electrodes. In some embodiments, cap 217 comprises less than 256 electrodes.

Electrodes 219 are positioned to contact a selected set of spatial scalp locations. In some embodiments, cap 217 comprises a mechanism 221 for re-positioning the electrodes so that the same electrodes contact a different set of spatial scalp locations. Additionally or alternatively, cap 217 is configured to be rotated with respect to the patient's scalp, so as to re-position the electrodes on the scalp. In an example, electrodes 219 record from a first set of spatial scalp locations during a first time period; then, either the cap is rotated and/or mechanism 221 moves the electrodes (e.g. rotates a frame onto which the electrodes are attached) into contact with a second set of spatial locations, and electrical brain activity is recorded by the re-positioned electrodes during a second time period.

In some embodiments, the cap or frame are constructed to fit the patient's head even when rotated, for example, leaving the eyes and/or ears of the patient uncovered. In an example, a cap configured to be axially rotated with respect to the patient's head (for example a 90 degree rotation, 180 degree rotation or intermediate, larger or smaller angles) would be shaped with relatively short temporal extensions so that rotation of the cap does not cover the patient's eyes.

System Components and Activation

In some embodiments, mechanism 209 and/or mechanism 221 are controlled by a controller 225. In some embodiments, the recorded data is transferred to a processor 227 which is configured for analyzing and integrating data recorded during the plurality of sessions. In some embodiments, results of the analysis and/or raw recorded data and/or patient details and/or other related information are transferred to a remote server 229. Optionally, data is stored in memory 231. In some embodiments, data is transferred to a physician and/or clinic. Optionally, the physician performs an assessment of the monitoring process and/or an assessment of the patient's condition based on the data received.

In some embodiments, controller 225 controls activation and/or positioning of the cap electrodes according to data analyzed by processor 227. In an example, controller 225 modifies a length of a recording session in response to indications received from the processor 227 (for example, recording is stopped if sufficient data had been obtained). In another example, controller 225 instructs to adjust electrode position and/or strengthen electrode attachment if a recorded signal is too noisy, and/or if an electrode was not accurately placed. Optionally, controller 225 controls recording based on instructions received from the physician, researcher, clinic, external database, remote server and/or other.

In some embodiments, the system comprises a user interface 233. The user interface may include (or be configured as) a dedicated cell phone application, a computer program, a screen (optionally touch screen) on the cap itself, a remote device in communication with the cap and/or other any other device or tool suitable for displaying data to the user and/or obtaining input from the user. In some embodiments, recorded data is displayed as a continuous flow, locally (e.g. to the user and/or caregiver) and/or remotely (e.g. to the physician, clinical center, or other).

EEG Electrodes

In some embodiments, electrodes of the cap (such as 203+205, 219) are dry electrodes. Some potential advantages of dry electrodes may include: facilitating attachment through hair; avoiding the need for conductive glue, avoiding the need for skin preparation, increasing a likelihood of consistent electrode placement; avoiding the risk of conductive bridges between electrodes; facilitating placement of a plurality of electrodes simultaneously.

Optionally, if dry electrodes are used, the electrodes are required to come in pressurized contact with the scalp. Therefore, in some embodiments, when using dry electrodes, a time period in which the dry electrodes come into operable contact with certain scalp locations may be limited to, for example, 15 minutes, 30 minutes, 1 hour or intermediate, longer or shorter time periods.

Alternatively, electrodes of the cap (such as 203+205, 219) are wet electrodes. Optionally, the wet electrodes are pasted to the scalp using a conductive glue (gel, paste and the like). In some embodiments, light abrasion of the scalp is performed before electrode pasting.

In some embodiments, a wet electrode is provided with a pre-prepared conductive glue pad attached to it. Optionally, prior to each use, the pre-prepared electrode and pad are attached to the cap at designated electrode locations. Then, right before attachment to the scalp, the glue pad is exposed (e.g. by peeling a cover). Placing of the cap on the scalp attaches and presses the electrodes and respective glue pads or conductive gel against the scalp to ensure contact.

Optionally, if wet electrodes are used, the electrodes may remain attached to the scalp for a relatively long period of time, for example for 6 hours, 1 day, 2 days, 3 days or intermediate, longer or shorter time periods.

Other System Parameters

In some embodiments, the system is operated via mains power. Additionally or alternatively, the system is battery operated. Optionally, the battery is rechargeable.

In some embodiments, a sampling rate of the signals is between 250 Hz to 10 KHz, such as 700 Hz, 1 KHz, 5 KHz or intermediate, higher or lower rates.

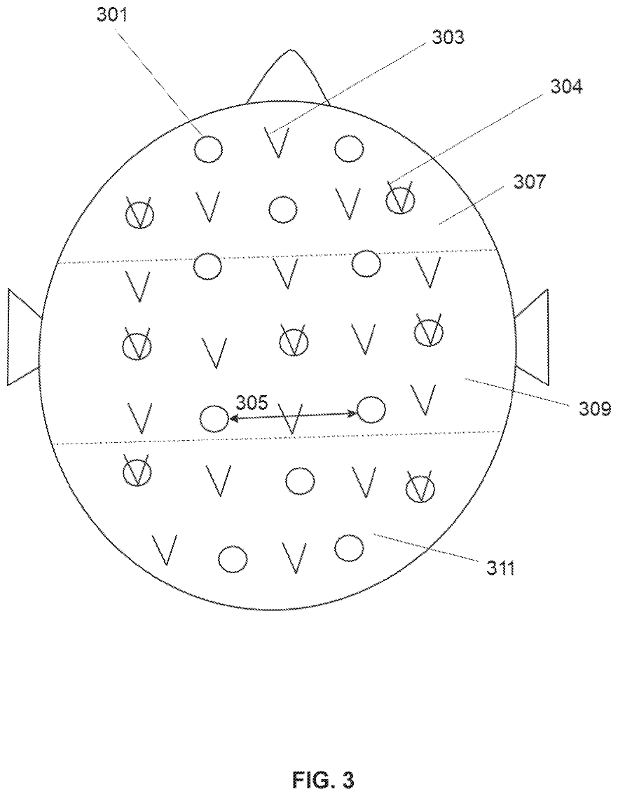

FIG. 3 schematically illustrates electrode positions at selected spatial scalp locations during different recording sessions, according to some embodiments.

In some embodiments, during different recording sessions, EEG signals are recorded from different sets of spatial scalp locations. In some embodiments, during the full monitoring period, recording is performed from two or more sets of spatial scalp locations in an alternating manner (e.g.: first set of locations for a first time session; second set of locations for a second time session; first set of locations for a third time session; second set of locations for a fourth time session, and so on).

In the example shown herein, a first set of electrodes 301 (indicated by circles) are positioned to record from a first set of spatial locations during a first time period; and a second set of electrodes 303 (indicated by the V marks) are positioned to record from a second set of spatial locations during a second time period.

In some embodiments, certain spatial locations are recorded from during both sessions (indicated by the dual circle-V mark, 304). In some embodiments, a number of “shared” spatial positions (which are recorded from during two or more sessions) is between 2-5 spatial positions, 3-7 spatial positions, 5-10 spatial positions, 3-20 spatial positions or intermediate, larger or smaller number. In some embodiments, the number of shared spatial positions is selected to be sufficient for ensuring that the electrographic entity is the same one. A potential advantage of recording from the same spatial scalp location during different (e.g. two or more) recording sessions may include improving detection of EEG waveforms during analysis of the recorded data.

In some embodiments, in one or more scalp areas which are of interest electrodes may be positioned at a higher density as compared to other scalp regions. In an example, if the patient is already suspected to have predominant right temporal spikes, electrodes at a relatively high density can be positioned at the right temporal region. Optionally, electrodes at a region of interest are at least 25%, 50%, 75% or intermediate, higher or smaller percentage denser as compared to other scalp regions. Optionally, a physical dense array is used for measuring from a scalp region of interest, while a virtual dense array is used for measuring from other scalp regions. A potential advantage of a denser array and/or a higher number of electrode positions that are shared between different recording sessions may include the ability to compare high spatial frequency components of the signals (in addition to the low spatial frequency components of the signals, for which sparse electrode positioning is sufficient).

In some embodiments, a distance 305 between adjacent electrodes of each of the sets ranges between, for example, between 0.5 cm to 10 cm, such as 1 cm, 4 cm, 8 cm, or intermediate, longer or shorter distances. *.

In some embodiments, each electrode set comprises between 8-24, 16-128, 36-256, or intermediate, larger or smaller number of electrodes for recording from a corresponding number of spatial scalp locations.

In some embodiments, electrodes of each set are distributed such that at least 1 or 2 electrodes are located in each of the forelock 307, midscalp 309 and crown 311 regions of the scalp. Optionally, between 4-60, 10-80, 5-30 or intermediate, larger or smaller number of electrodes are distributed on each region.

In some embodiments, electrodes are distributed such that at least 1-20 electrodes are positioned along T1/T2 (temporal) positions and along the sub-temporal chain. In some embodiments, the position is T1. In some embodiments, the position is T2.

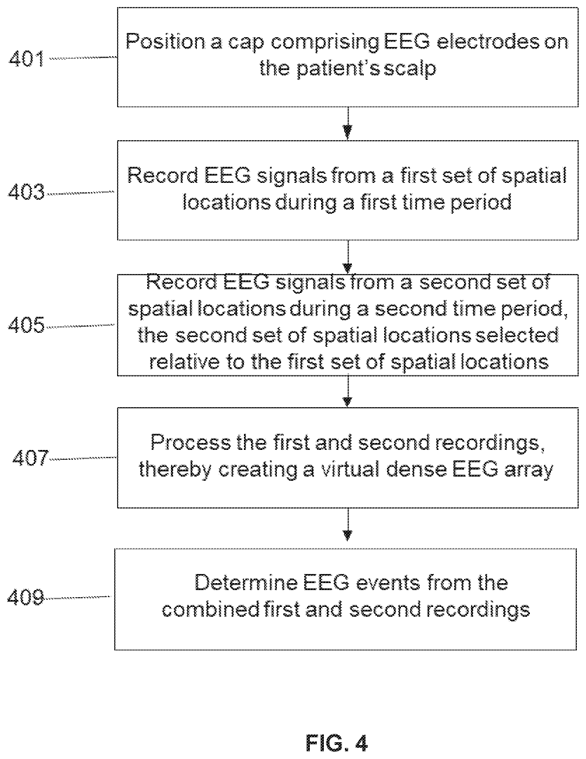

FIG. 4 is a flowchart of a method for EEG monitoring by creating a virtual dense array of EEG electrodes, according to some embodiments.

In some cases, EEG monitoring using a dense array of electrodes is advantageous, for example for the purpose of accurate localization of the source of the signal. The flowchart of FIG. 4 describes a general method for creating a virtual dense EEG array by recording data during a plurality of sessions, whereby in each session only a limited number of sparsely placed electrodes is used, in accordance with some embodiments.

In some embodiments, a cap comprising EEG electrodes is positioned on the patient's scalp (401). During a first time period, EEG signals are recorded from a first set of spatial scalp locations (403), sparsely distributed across the scalp. Then, by displacing the electrodes relative to the first set of spatial scalp locations and/or by moving a different set (or only partially overlapping set) of electrodes into operable contact with the scalp, a second set of spatial scalp locations is contacted. EEG signals are then recorded from the second set of spatial locations during a second time period (405).

Optionally, the process is repeated during a complete monitoring period, alternating between the different sets of spatial scalp locations. It is noted that in some embodiments, more than 2 sets may be defined, for example, 3, 4, 5, 6, 10 or intermediate, larger or smaller number of different sets of scalp locations are being recorded from during different time periods.

In some embodiments, the recording sessions are performed in a continuous manner; alternatively, there may be recesses between the sessions. Optionally, monitoring is stopped for a certain time period and then continued, for example to allow the patient to perform certain activities, optionally without the cap on (shower, sleep, or others).

In some embodiments, data recorded during the sessions (for example during a first and second sessions) is processed to thereby create a virtual dense EEG array (407). In some embodiments, processing involves interpolating data recorded from the two sets of spatial scalp locations.

In some embodiments, EEG events are determined from the interpolated data (409). Optionally, using the interpolated data, irritative brain zones can be identified, and source electrical activity can be localized.

In some embodiments, combining the data recorded during the different sessions results in a virtual dense array, imitating a higher spatial resolution than the actual sparse resolution of each of the different sessions.

In some embodiments, data is recorded by two or more relatively sparse arrays, and then interpolated to be equivalent to a denser array. In an example, two sparse arrays each including 32 electrodes are used for recording at two different time sessions, and then interpolated, the result being equivalent to a denser array of 64 electrodes.

Some embodiments may involve reconstruction of data, for example computative reconstruction of data for “missing” electrodes, for example as described in Lantz, G., De Peralta, R. G., Spinelli, L., Seeck, M., & Michel, C. M. (2003): “Epileptic source localization with high density EEG: how many electrodes are needed?” (Clinical neurophysiology, 114(1), 63-69).

Some potential advantages of constructing a virtual dense array in which the actual measurement at any given time requires use of only a sparse, limited number of electrode positions may include: reducing or avoiding the formation of conductive bridges between adjacent electrodes (due to that electrodes may be more spaced apart from each other as compared to an actual dense array); simplifying use (e.g. attachment of electrodes to the scalp); and/or increasing the patient's comfort as less scalp locations are being contacted (and in some cases pressurized, for example if dry electrodes are used).

FIGS. 5A-B are a flowchart of a method for processing EEG signals obtained by two different sparse electrode arrays at two different time instances (5A) and a detailed flowchart of interpolation of data recorded at the two different time instances (5B), according to some embodiments.

In some embodiments, for example as described hereinabove, EEG is recorded using two sets of sparse electrode positions at two different time instances (501). (In some embodiments, measuring is performed with more than two sets).

In some embodiments, processing is based on that neuro-electrographic events are non-homogenous in their spatial characteristics. Distribution of the electric field through the scalp is represented by a set of components with different spatial frequencies.

In some cases, sampling of low spatial frequencies, for example in the range of 0.1-0.25 cycles/cm, can be achieved using an electrode array with a relatively low spatial density, while sampling of high spatial frequencies, for example in the range of 0.26-0.4 cycles/cm requires a spatially denser electrode array. Other ranges for example as described in Freeman, W. J., Holmes, M. D., Burke, B. C., & Vanhatalo, S. (2003). “Spatial spectra of scalp EEG and EMG from awake humans”. Clinical Neurophysiology, 114(6), 1053-1068 may also be relevant. Commonly, sources of electrical brain activity that are closer the head surface such as the Dorsolateral cortex have higher spatial frequencies than deep brain sources such as the Mesial or basal cortex.

When recording using two sparse electrode arrays, for example as described herein, similar types of EEG events may be recorded at two different time instances. Therefore, in some embodiments, processing is performed by clustering low spatial frequency components of the recorded EEG events according to their temporo-spatial characteristics (503). Inside each clustered group, electrode positions are interpolated to generate a virtual dense array (505). An example of an interpolation process is described in FIG. 5B, further detailed below. In some embodiments, clustering into groups and subsequent averaging inside each of the groups employs method such as primary component analysis (PCA), independent component analysis (ICA), and/or other machine learning, pattern recognition and/or data mining methods for correlating between variables are implemented.

In some embodiments, high spatial frequency components of the recorded EEG events are revealed from the virtual dense array that was generated (507). Optionally, temporo-spatial characteristics of the high spatial frequency components can then be studied. By revealing the high spatial frequency components, superficial sources of the electrical brain activity (e.g. in the dorsolateral cortex of different lobes: frontal, parietal, temporal and/or occipital) can be detected.

The flowchart of FIG. 5B is an example of a method for generating a virtual dense array by interpolating spatial locations of the two different sets, inside each of the clustered groups of low spatial frequency components. In some embodiments, a local electric field recorded by each electrode of each of the two arrays is represented by a 4-dimensional vector (505A), including Cartesian coordinates of the electrode location, and the recorded amplitude ([x, y, z, amplitude]). For each of the two sparse arrays, a 4×n matrix is constructed, n representing the number of electrodes in the array (505B). An example of a matrix representing the sampled field of a single array comprising “n” electrode positions:
[x 1, y 1, z 1, amplitude 1;
x 2, y 2, z 2, amplitude 2;
. . . ;
x n, y n, z n, amplitude n]
   Matrices of the two arrays (referred to as array A, array B) are then integrated, producing a 4×2n matrix (505C), represented for example as follows:
[x 1_A, y 1_A, z 1_A, amplitude 1_A;
x 1_B, y 1_B, z 1_B, amplitude 1_B;
x 2_A, y 2_A, z 2_A, amplitude 2_A;
x 2_B, y 2_B, z 2_B, amplitude 2_B;
. . . ;
x n_A, y n_A, z n_A, amplitude n_A;
x n_B, y n_B, 1_B, amplitude n_B]

In some embodiments, a virtual electric field represented by an integrated matrix takes into account electrode positions and recorded amplitudes of both arrays. In some embodiments, electrode positions are represented using a polar coordinate system.

In some embodiments, the scalp is represented by planar coordinates, and electrodes positions are represented in a two-dimensional system (using only xy coordinates).

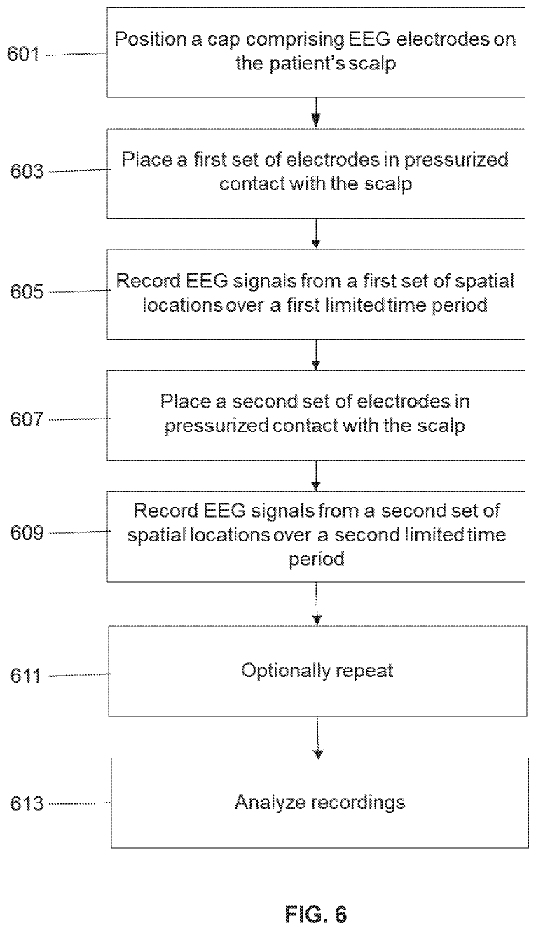

FIG. 6 is a flowchart of a method for EEG monitoring in which electrodes are placed in contact with the scalp for a limited time period, according to some embodiments.

The following method may be especially advantageous with the use of dry EEG electrodes, which need to form pressurized contact with the scalp for properly recording the electrical activity.

In some embodiments, a cap comprising EEG electrodes is positioned on the patient's scalp (601). To begin measurement, a first set of electrodes forms pressurized contact with the scalp (603). In some embodiments, initial positioning of the cap automatically places the first electrode set at recording positions; additionally or alternatively, the electrodes are moved (e.g. rotated, pushed, slid) into pressurized contact with the scalp, for example via a mechanical, electromechanical, hydraulic, pneumatic and/or other cap mechanism suitable for moving selected single electrodes and/or an electrode frame or channel into pressurized contact with the scalp.

In some embodiments, during a first limited period of time, for example no longer than 15 minutes, 30 minutes, 45 minutes, 1 hour, 2 hours, EEG recording is performed using the first electrode set (605).

Optionally, at the end of the first recording session, the first set is moved away from the scalp (e.g. lifted away), and a second set is moved into pressurized contact with spatial scalp locations different than those contacted by the first electrode set (607). In some embodiments, there is at least some overlap between lifting of the first set and positioning of the second set in contact with the scalp. A potential advantage of such overlap may include facilitating the determining of relative electrode positions between the first set and the second set.

In some embodiments, EEG is recorded by the second set over a second limited period of time (609). Optionally, the process described in 603-609 is repeated throughout a complete monitoring period, alternating between the different sets.

The recorded data is then analyzed (613), for example using methods as described herein.

Some potential advantages of limiting a time period in which the electrodes come into pressurized contact with the scalp may include reducing discomfort to patient; providing for a prolonged total monitoring period, for example days (by alternating spatial locations); reducing pain (e.g. reducing pressure sores) and/or skin damage to the scalp.

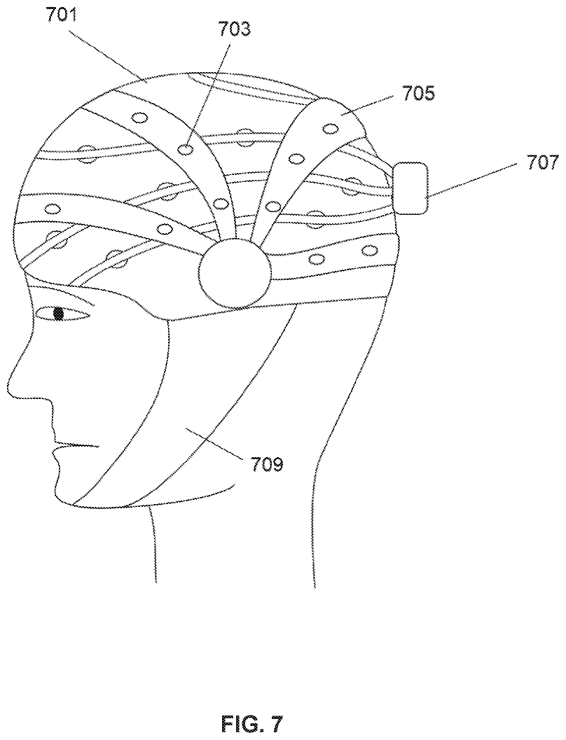

FIG. 7 is a schematic illustration of a cap comprising a mechanism for positioning selected electrodes against the scalp, according to some embodiments.

In some embodiments, cap 701 comprises a plurality of electrodes 703 embedded within and/or mounted onto a plurality of flexible, adjustable elements such as straps or bands 705. In an example, the electrodes are arranged on a band in a serial manner with a distance of between 3 mm-6 cm, 10 mm-2 cm, 1 cm-5 cm or intermediate, longer or shorter distances between adjacent electrodes. In some embodiments, bands 705 extend across the scalp, for example along or parallel to a posterior-anterior axis of the scalp, along or parallel to a transverse axis of the scalp, and/or other any other directions.

Moving the electrodes into operable contact with the scalp may be performed in several manners or a combination thereof. In some embodiments, each of bands 705 is individually tensioned (e.g. by being pulled on). In some embodiments, the band comprises a sleeve or tube which is inflatable (e.g. with fluid or air), whereas inflation moves the mounted electrodes into contact with the scalp, and deflation may lift the electrodes away from the scalp. Additionally or alternatively, electrodes are embedded within channels extending within the cap (not shown). Optionally, bands 705 are positioned above the channels and upon tensioning the bands the electrodes are pressed against the scalp.

In some embodiments, actuation of the band adjustment is carried out via one or more engines 707. In some embodiments, an engine 707 is configured to set tension to the bands, in an individual manner (per each band) or by simultaneously setting tension to a set of bands which comprises for example a first set of electrodes. In some embodiments, engine 707 is a hydraulic or pneumatic engine (e.g. a compressor), which pumps air or fluid into inflatable or fillable bands. Optionally, the fluid includes oil and/or water.

In some embodiments, cap 701 comprises one or more straps 709 for restraining the cap to the patient's head and optionally for adjusting a fit. In an example, strap 709 is a chin strap (similar to a bicycle helmet strap).

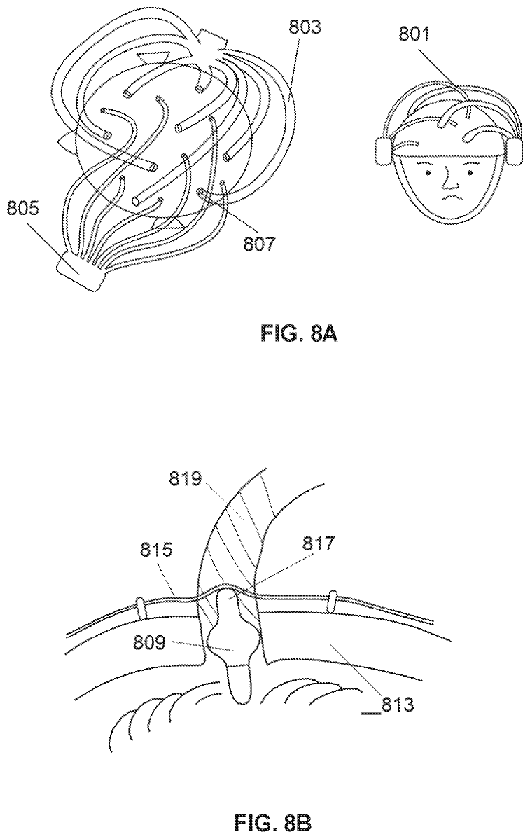

FIGS. 8A-B are schematic illustrations of a cap comprising hydraulic or pneumatic tubes for positioning electrodes against the scalp (8A) and a schematic drawing of a single electrode being pressured against the scalp (8B), according to some embodiments.

FIG. 8A schematically illustrates a cap 801 comprising or in some embodiments composed of a plurality of inflatable tubes 803. In some embodiments, tubes 803 extend from one or more engines 805, for example located at a most posterior point of the cap, at transverse side points of the cap, at an anterior point of the cap and/or other locations. Optionally, a tube extends between opposing engine locations. When a tube is inflated, the one or more electrodes 807 positioned along that tube are brought into operable contact with the scalp, and are optionally pressured against the scalp in response to increased inflation pressure.

FIG. 8B schematically illustrates an electrode 809 positioned in contact with the scalp 811. In the example shown, electrode 809 is held by a frame 813, optionally comprising a pressing band 815 which extends across a distal end 817 of electrode 809 and is tensioned so as to push down on the electrode. In some embodiments, electrode 809 is moved into contact with the scalp in response to inflation of a tube 819 (e.g. with air or other fluid). In some embodiments, tube 819 functions in a syringe like manner, and electrode 809 is positioned within the tube such that it can travel distally away from the scalp (e.g. in response to suction of air or fluid from the tube) or proximally towards the scalp (e.g. in response to filling air or fluid into the tube).

In some embodiments, the cap may comprise plurality of inflatable chambers. Optionally, inflation of a chamber moves one or more electrodes mounted with respect to the chamber to a position in which the electrode(s) contact the scalp. In some embodiments, inflation and/or deflation of chambers is performed selectively, for example some chambers are inflated while other are deflated.

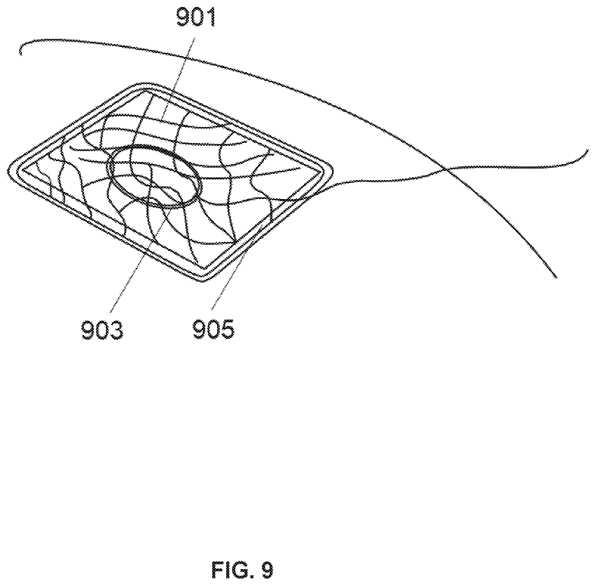

FIG. 9 is a schematic illustration of an integrated electrode and conductive glue pad, according to some embodiments.

In some embodiments, especially in which wet electrodes are used, attachment of an electrode to the scalp involves pasting a conductive paste or glue (e.g. collodion) to adhere the electrode to the scalp (optionally through hair) and maintain the electrode affixed to the scalp at the desired position.

The integrated electrode and glue pad illustrated in FIG. 9 comprises, according to some embodiments, a flexible glue pad 901 onto which an electrode 903 is mounted. In some embodiments, the pad 901 is surrounded by a frame 905.

Optionally, frame 905 is plastically deformable so that it can be shaped by a user, for example to conform to the curvature of the scalp at the scalp area selected for electrode attachment. In some embodiments, frame 905 is yet rigid enough to prevent the pad from twisting, folding onto itself, and/or otherwise interfering with direct attachment of the electrode to the scalp. In some embodiments, the frame prevents from pad portions from sticking to each other.

Some potential advantages of an integrated pad and electrode may include facilitating and simplifying electrode attachment (for example such that the process can be carried out by a caregiver, as opposed to requiring the skills of a professional EEG technician; reducing the time it takes to adhere the electrodes to the scalp; and potentially simplify removal of the electrode from the scalp at the end of use, for example by gripping the frame to peel the electrode away from the scalp.

Optionally, frame 905 is made of plastic.

In some embodiments, an adhesive side of the pad comprises a pull-off paper (like a sticker) or a gauze.

In some embodiments, a set of ready to stick electrodes are embedded in a cap or frame. Additionally or alternatively, ready to stick electrodes are placed directly on the scalp, without a holding frame or cap.

In some embodiments, the glue pad is filled by a conductive paste (e.g. 110-10 type paste, which is conductive and adhering). Alternatively, the glue pad comprises only an adhering material, and additional conductive gel is applied.

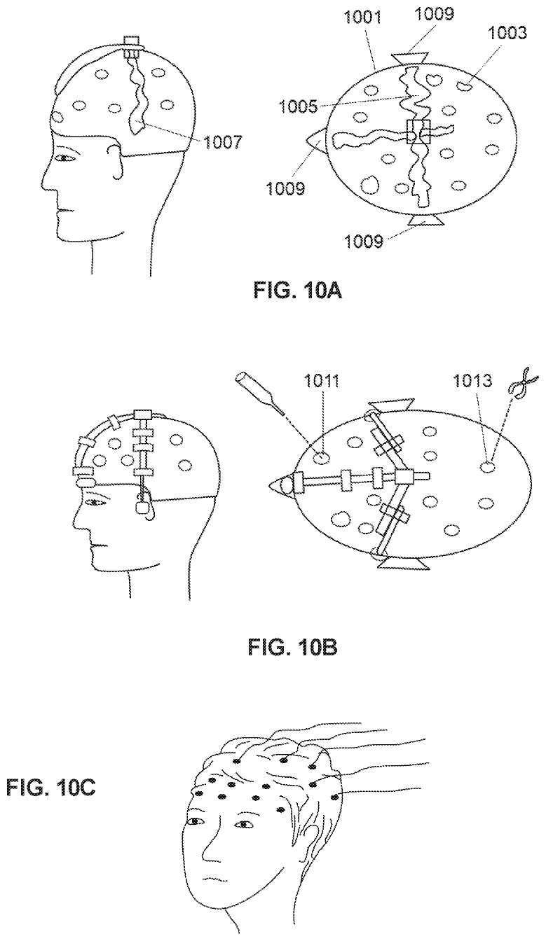

FIGS. 10A-C illustrate a method for measuring and marking electrode positions on the patient's scalp, according to some embodiments.

The method described in FIGS. 10A-C may be implemented for initial fitting of a cap to a patient, and may be carried out by a professional technician or physician, or, alternatively, by a caregiver.

FIG. 10A schematically illustrates fitting of a model cap 1001 which comprises a plurality of openings 1003 at electrode positions.

In some embodiments, a size of the model cap is selected to be similar to a structured cap which is used during EEG-fMRI monitoring, for example so that electrode positions will be matching. Matching electrode positions between the two modalities (EEG-fMRI and long term EEG) may provide for facilitating integration and/or comparison of the data.

In some embodiments, cap 1001 comprises a set of straps or strings 1005, extending for example along or parallel to a posterior-anterior axis of the cap, and along or parallel to a transverse axis of the cap. Optionally, the strings are adjustable but are not elastic, so that their length remains constant. During fitting of the cap, loose ends 1007 of the strings are pulled on in order to approximate the cap to the scalp, such that the strings travel the shortest distance across the cap. Then, the loose ends of the strings are attached (e.g. using glue, a clip, or other attachment means) to anatomical landmarks 1009. For the purpose of approximating the cap to the scalp, following stretching of the string, the string may be cut shorter, or knotted to another string or string portion thereby producing an overlap between the strings.

In some embodiments, once an initial fit has been obtained, the model cap may be removed from the patient's head. When the model cap is placed back on the patient's head, by ensuring that the strings are stretched and the ends of the strings are attached to the anatomical landmarks, openings 1003 of the cap are ensured to be placed at similar scalp locations as they were before, marking constant electrode positions.

In some embodiments, electrodes are then attached via the openings, or, alternatively, electrode positions are marked via the openings (for example by marking the scalp with colored dots 1011 and/or by placing clips 1013 on hair adjacent the opening), the cap is removed, and electrodes are pasted according to the marking. FIG. 10C schematically illustrates the final electrode arrangement on the scalp.

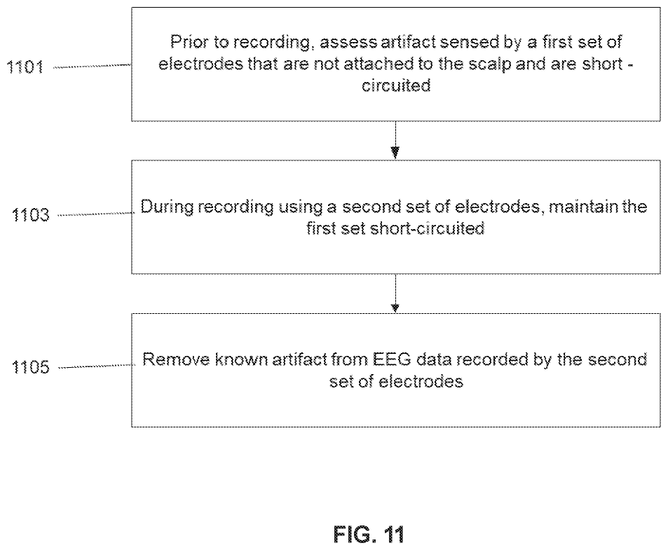

FIG. 11 is a flowchart of a method for reducing noise from an EEG recording, according to some embodiments.

A method as described herein may be implemented in situations in which, at a given time, not all electrodes are used for recording. Optionally, not all electrodes are in contact with the scalp.

In some embodiments, prior to EEG recording, electrodes of each alternating set are short circuited, for example by producing loops between electrode pairs of a set. Since the short-circuited electrodes are sensitive to external electro-magnetic fields in the surroundings of the patient and/or are sensitive to movement, the short-circuited electrodes can function as artifact sensors.

In some embodiments, an artifact sensed by the short-circuited electrodes is detected and assessed (1101). Then, during EEG monitoring via a second set of electrodes (which are attached to the scalp), the short-circuited electrodes remain as such (1103). Once a recording session via the second set of electrodes had been completed, the short-circuited electrodes are disconnected from each other and moved to a recording position, in contact with the scalp, while the second set of electrodes (which was optionally lifted away from the scalp) is now paired to be short circuited. In some embodiments, altering between the electrode sets and pairing of electrodes of the set which is currently not in contact with the scalp are carried out via suitable circuitry, including for example a switch or relay element. In some embodiments, short-circuiting electrodes is performed manually.

In some embodiments, during processing of the data recorded, the pre-known artifact sensed by the looped electrodes can be removed, thereby potentially increasing the signal to noise ratio.

Other signal to noise improvement methods may include: averaging signal amplitudes recorded by an electrode set and/or by two or more specific electrodes; independent component analysis (ICA); principle component analysis (PCA); band pass filtering and/or other suitable noise reduction methods.

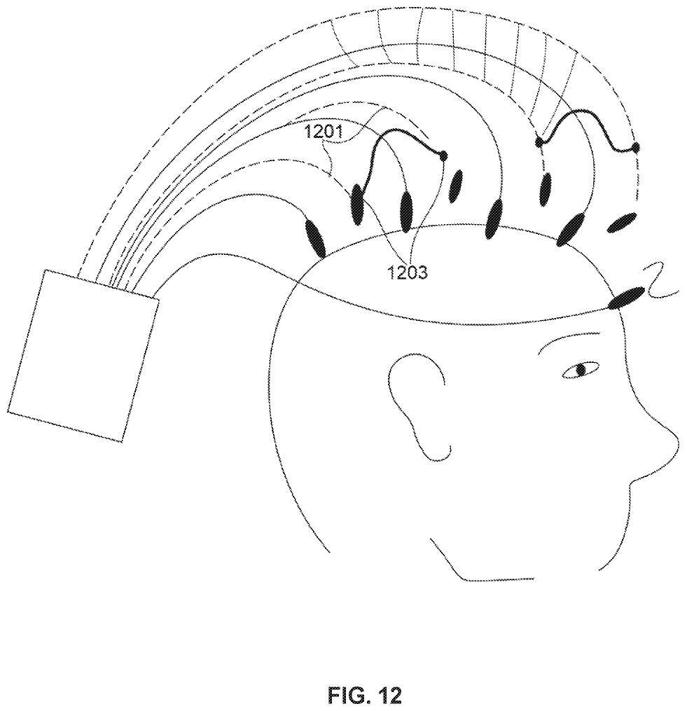

FIG. 12 schematically illustrates pairing of electrodes in a cap comprising dry electrodes, according to some embodiments. In some embodiments, electrode loops are produced by electrically connecting between wires 1201 of a pair of electrodes 1203. Optionally, electrode pairing is performed for all electrodes of a set which is currently not in contact with the scalp, for example lifted away from the scalp as shown.

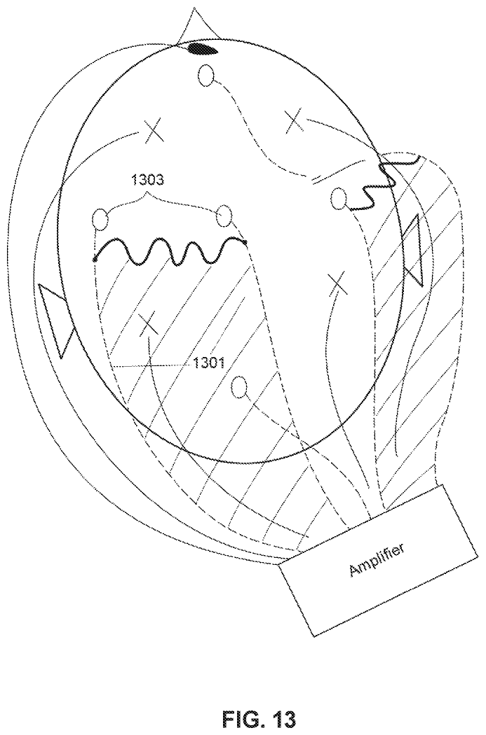

FIG. 13 schematically illustrates pairing of electrodes in a cap comprising wet electrodes, according to some embodiments. In some embodiments, electrode loops are produced by electrically connecting between wires 1301 of a pair of electrodes 1303. Optionally, when wet electrodes are used, conductive glue or gel is transferred (e.g. pumped) to fill electrodes used for a current recording session, while the other electrode set (which may or may not remain in contact with the scalp, but does not comprise conductive glue) is short circuited.

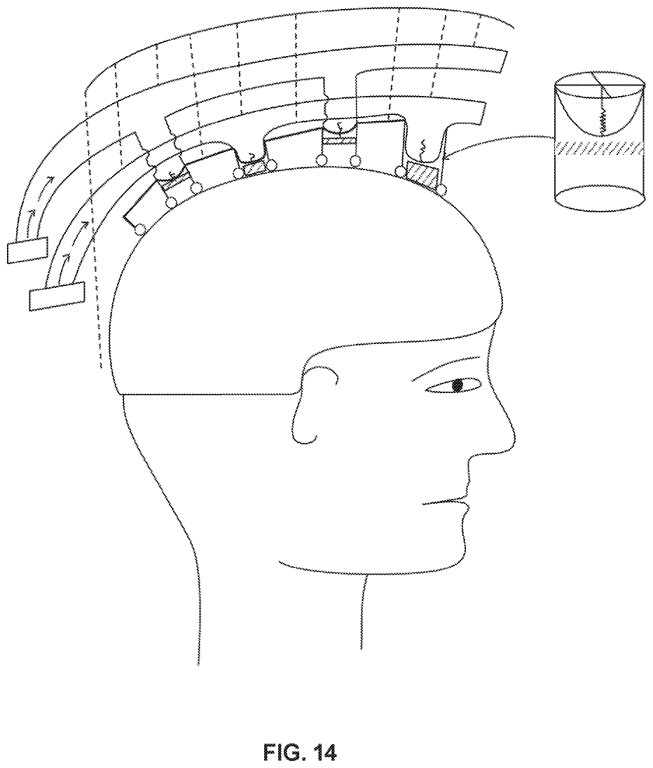

FIG. 14 is a schematic illustration of a cap 1400 comprising a spring-actuated mechanism for mounting and/or dismounting the EEG electrodes on the scalp, according to some embodiments.

In some embodiments, electrodes 1401 are each positioned with respect to an elastic element such as a spring 1403.

In some embodiments, the electrode and spring assembly is located in a designated channel 1405 which is in fluid communication with an inflatable or fillable tube 1407. In some embodiments, tube 1407 extends across the scalp, such as from a posterior position to an anterior position, and/or from a first temporal position to a second temporal position. Optionally, a plurality of electrode channels such as 1405 are distributed along tube 1407. In some embodiments, tube 1407 comprises an elastic lining 1409, for example an elastic film, which extends into the channel such that a proximal end of spring 1403 is mounted onto the elastic film or positioned against it.

In some embodiments, in use, inflating or filling of tube 1407 (e.g. via a pump 1411, for example with fluid and/or gas, such as air) causes lining 1409 to extend proximally within channel 1405, pushing down on electrode 1401 to position the electrode in contact with the scalp. Optionally, in this position, spring 1403 is stretched by the movement of lining 1409 and tensioned. In some embodiments, to lift the electrode from the scalp, deflation of tube 1407 (e.g. via pump 1411) creates suction which pulls the lining back towards the tube, thereby pulling on spring 1403 which bounces back and retracts electrode 1401 along with it distally away from the scalp.

In some embodiments, electrode sets are arranged such that inflation of a first tube 1407 places a first set of electrodes against the scalp, and inflation of a second tube 1413 places a second set of electrodes against the scalp, optionally while the first set was retracted in response to deflation of the first tube.

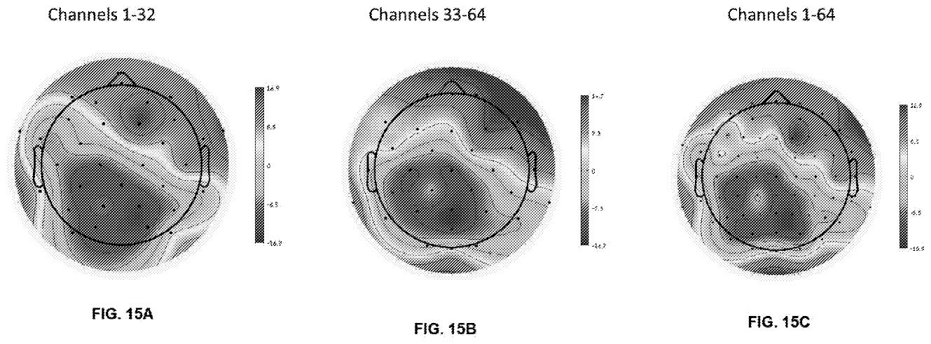

FIGS. 15A-C are graphical examples of an electric field recorded by a 64 channel EEG array (FIG. 15C) and electric fields constructed from data of two subsets of electrode arrays, each including 32 electrodes (FIG. 15A, FIG. 15B), according to some embodiments.

In some embodiments, the higher density of contour lines at certain scalp regions is indicative of a faster change in the measured amplitudes, associated with higher spatial frequency components. As can be observed from this example, generating an electric field based on a subset of 32 electrodes is characterized by contour lines of reduced density, as compared to the full 64 electrode array, yet, the existing contour lines are still indicative of lower frequency components. Therefore, in some embodiments, interpolating electric fields obtained by two different subsets of electrodes may provide for generating a higher resolution electric field, which is indicative of low as well as high spatial frequency components.

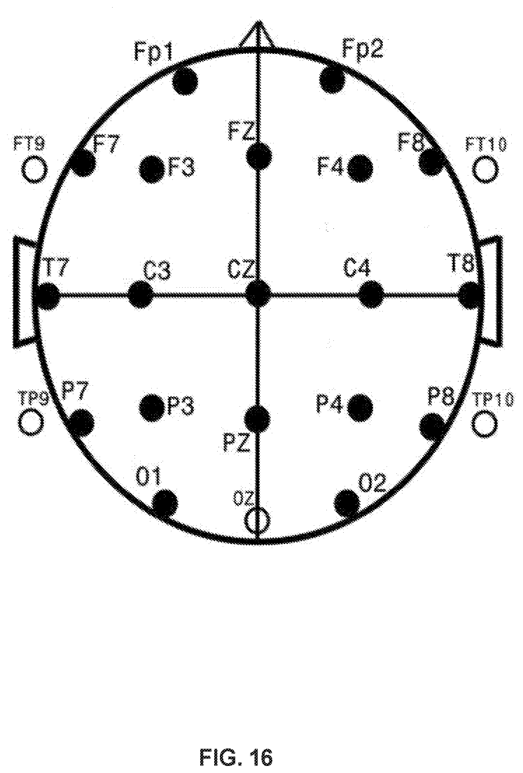

FIG. 16 is an example of a sparsely distributed electrode array on the scalp, according to some embodiments. In this example, the array comprises 24 electrodes, each electrode marked according to its spatial scalp position. In this example, electrodes are symmetrically distributed, for example relative to the long and transverse axis of the scalp.

It is noted that other arrangements are also contemplated, including various numbers of electrodes (e.g. 4, 8, 16, 28, 32, 45, 72, 96, 120, 200 electrodes) or intermediate, higher or smaller number of electrodes. It is noted that various spatial distributions are also contemplated, including patterned and/or randomly distributed electrodes. Some embodiments may include asymmetric distributions.

FIG. 17 illustrates a simulation performed in accordance with some embodiments, simulating EEG recording at 4 different time instances using a sparse electrode set of 16 electrodes, and interpolating the recordings to obtain a measurement equivalent to one performed by use of 64 electrodes simultaneously. The simulation was performed by monitoring EEG using a set of 64 electrodes, during which a total of 536 interictal epileptic discharges (IEDs) were recorded. A distribution of the averaged amplitudes of the IEDs obtained by the 64 electrode set is shown at 1701. To simulate a measurement by a set of fewer electrodes, the total of IEDs (536) was randomly divided into 4 groups, represented by 1703, 1705, 1707, 1709, each simulating a distribution of averaged amplitudes of 134 different IEDs, recorded by a set of 16 electrodes (channels) only, at a certain time instance. Data of the 4 groups was then interpolated, as shown at 1711. It can be observed that the distribution constructed by integration of the data of the 4 groups, at 1711, is substantially equivalent to the original distribution obtained by the actual measurement using the 64 electrodes set.

It is noted that while the simulation described herein was performed by integration of 4 different sets, other embodiments may involve integration of, for example, only 2 sets, 3 sets, 5 sets, 7 sets, 10 sets, or intermediate, larger or smaller number of different electrode sets, each set optionally being placed at different spatial scalp locations.

In some embodiments, the EEG waveforms (EEG frequency patterns) may be selected from: Alpha waves (Alpha rhythm), Beta waves (b-waves), Theta waves and Delta waves. Each possibility is a separate embodiment. In some embodiments, Beta waves are in the frequency of 14-30 Hz. In some embodiments, Alpha waves are in the frequency of 8-13 Hz. In some embodiments, Theta waves are in the frequency of 4-7 Hz. In some embodiments, Delta waves are in the frequency of 1-3 Hz.

During relaxed wakefulness the human brain exhibits several types of distinct rhythmic electrical activity in the alpha frequency band (8-13 Hz) in occipital, parietal, and central areas. These rhythms differ in topography, frequency, sensitivity to tasks. Frontal alpha asymmetry may serve as a neuromarker for depression and other conditions, such as anxiety. Alpha oscillations in the primary sensory areas may also be responsible for maintaining optimal level of functioning.

According to some embodiments, the methods, devices and systems disclosed herein may be used to reconstruct values of electric field generated by the brain. In some embodiments, the reconstruction of the values of the electric field generated by the brain, may be facilitated in terms of a virtual 32-electrode array, sampled by two sparce (16 electrodes) EEG arrays at different time points.

According to some embodiments, the separate EEG arrays may be facilitated by one cap, in which the location of the electrodes may be replaceable/adjustable as detailed herein. In some embodiments, the separate EEG arrays may each be located on a separate/individual cap (for example, two separate caps) that may be used interchangeably, whereby within each cap, at least some of the locations of the electrodes may be different.

In some embodiments, the EEG amplifier may use 19 channels (in addition to reference and ground) that may be sequentially or simultaneously connected to two electrode arrays.

In some embodiments, each electrode array (either individually placed/located on separate caps, or interchangeable on a single cap, as detailed above herein) may include: a designated number of EEG channels (for example, 16 channels) tested at different time points (each array includes at least partially different electrode location between different arrays), a spatial region electrode (for example, T1 electrode (for example, connected by a sticker to the left temple area), a reference electrode (for example, connected by sticker to right temple area (T2); ground electrode (for example, connected by sticker to a designated area (for example, an area just below and behind right mastoid); or any combinations thereof. Each possibility is a separate embodiment. In some embodiments, one or more (such as, two) Electrocardiogram (ECG) electrodes may further be connected to the chest of the subject (for each array testing).

According to some embodiments, the recorded signals may be transferred using a suitable amplifier to a processing unit (such as, computer, laptop, PC, tablet, and the like), by any suitable means, such as wired or wireless (for example, Bluetooth).

According to some embodiments, various types of EEG waves may be detected/measured. For example, awake EEG of a subject may be recorded. In some embodiments, other brain waves, such as, alpha rhythm may be recorded. In some exemplary embodiments, the subject produces posterior alpha rhythm brain waves (for example, by repeatedly opening and closing the eyes). In some embodiments, each EEG recording may be over a period of about 10 seconds to about 60 minutes.

In some embodiments, prior to recording of the electrical signals, conductive past may be placed between the EEG electrodes and the scalp skin.

According to some embodiments, analog signals may be digitized at any desired frequency, such as in the range of about 10 Hz-10 KHz. For example, the frequency may be of 500 Hz.

According to some embodiments, during the recording, the EEG signals may be visualized in real time. In some embodiments, the detected electrical traces may be processed and visualized using suitable software and algorithms. In some embodiments, the EEG data matrices generated may consequently be processed using a corresponding algorithm. In some embodiments, the processing includes at the first stage data files containing the EEG signals and thereafter, the EEG may be re-referenced to the average of all electrodes on the corresponding cap or array (when one cap is used with separate arrays). At the next step, the timings of the detected epochs (such as, for example, alpha rhythm epochs) may be recalculated from seconds to time-points, for example, by multiplying seconds by the sampling frequency. Next, statistical tools (such as, for example, Fast Fourier Transform (FFT)) may be utilized to calculate frequency spectrogram absolute value (intensity) and phase. In some exemplary embodiments, the statistical analysis may be applied to 1 sec (which may equal 500 timepoints) of the EEG after the beginning of epoch, such that the frequency resolution (bin width) is 1 Hz.

According to some embodiments, since signals recorded at different times may not necessarily maintain the same phase, to superimpose such signals in frequency domain, one of the signals is rotated and the optimal phase is selected. In some embodiments, this may be achieved by two steps: The real part of the 1st-bin (for example, 10 Hz bin) may be calculated by multiplying the absolute value of the 10-Hz bin to cosine of its phase. In some embodiments, the second array frequency data may be rotated by multiplying the absolute value by cosine of changing phase from 0 to pi radians—with the steps of 0.1 pi radians. Next, the values of interpolating points may be calculated for electrodes of first array in longitudinal direction. Since the electrodes of the second array may be located between electrodes of the first array in longitudinal direction, the field values in these interpolated points are expected to correspond approximately to values of the second array channels. Accordingly, the real part of the second array rotated FFT may be selected by maximal correlation with the first array) interpolated points vector. According to some embodiments, since the optimal phase between signals of two arrays is selected, the virtual dense array field may be reconstructed, providing that the signals measured by different arrays are the same power (or amplitude). Since the same brain generators at different type may exhibit different power, the signals measured at different time may be normalized according to their power.

According to some embodiments, as exemplified herein, the same type of EEG signal recorded by different sparce EEG arrays at different time points, can be used for denser electric field reconstruction and the constructing virtual EEG array.

According to some embodiments, posterior alpha rhythm may be sampled. In some embodiments, any various types of brain electrical signals may be sampled and analyzed by the methods disclosed herein, including, for example, different types of epileptiform EEG signals.

According to some embodiments, connecting different arrays (for example, in the form of different caps, or alternating arrays in a single cap, as detailed herein) to the same amplifier at different time points may be used chosen as a way of sparce EEG array electric field sampling. In some embodiments, as detailed above herein, the methods for virtual dense array EEG can include, for example, alternating electrode arrays, for example, by alternating pressure on dry or wet EEG electrode sets/arrays, such as the rising portions of the electrodes in the cap, disconnecting from the scalp, while pressing another electrode set to the scalp, alternating from time to time between the electrode sets, and the like, or combinations thereof. Each possibility is a separate embodiment.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1—Virtual Dense EEG Array Reconstruction

The aim of this experiment was to reconstruct values of electric field generated by the brain, in terms of a virtual 32-electrode array, while sampled by two sparce (16 electrodes) EEG arrays at different time points.

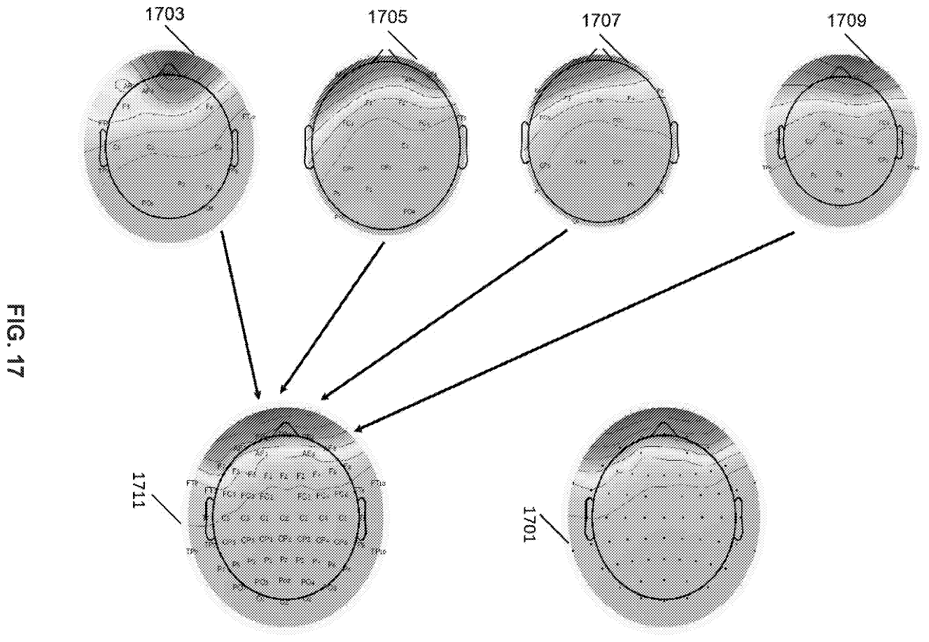

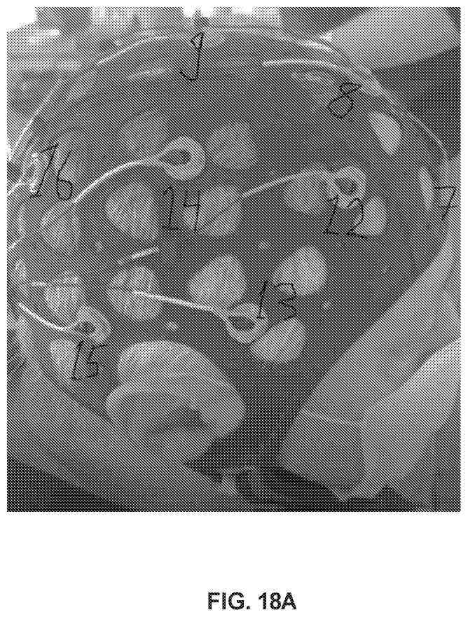

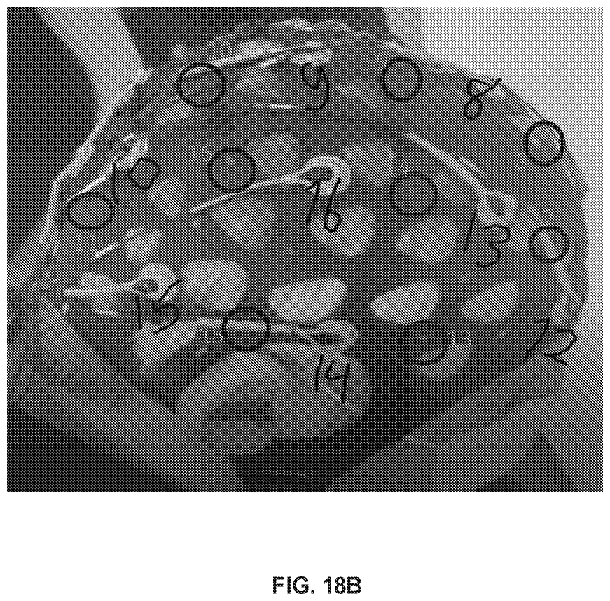

To this aim, the EEG device, specified for virtual dense array EEG, as disclosed herein was utilized. The EEG amplifier which uses 19 channels (in addition to reference and ground) was sequentially (not simultaneously) connected to two electrode arrays. Each array was composed by:
1. 16 Channel EEG array caps with different electrode locations (Cap 1 (shown in FIG. 18A) and Cap 2, (shown in FIG. 18B)), each of the caps placed on the patient head at a different time point.
2. T1 electrode connected by sticker to left temple area.
3. Reference electrode connected by sticker to right temple area (T2).
4. Ground electrode connected by sticker to the area just below and behind right mastoid.
5. Two ECG electrodes that were connected to the chest of the subject.

Amplifier transferred the signals to tabled computer by wireless connection using Bluetooth connection.

Awake EEG of the healthy adult subject was recorded by these two arrays at different times. Each EEG recording lasted several minutes. Before the recording Ten-20 conductive paste was placed between EEG electrodes and skin of the scalp. The subject was asked to close and open his eyes during EEG recording—this was aimed to produce posterior alpha rhythm.

The analog signals were digitized at frequency of 500 Hz.

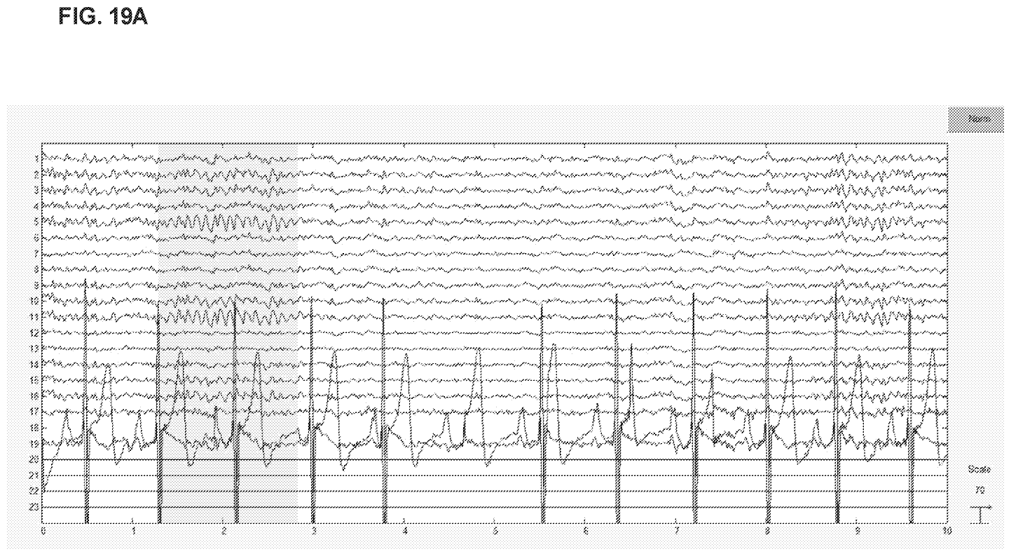

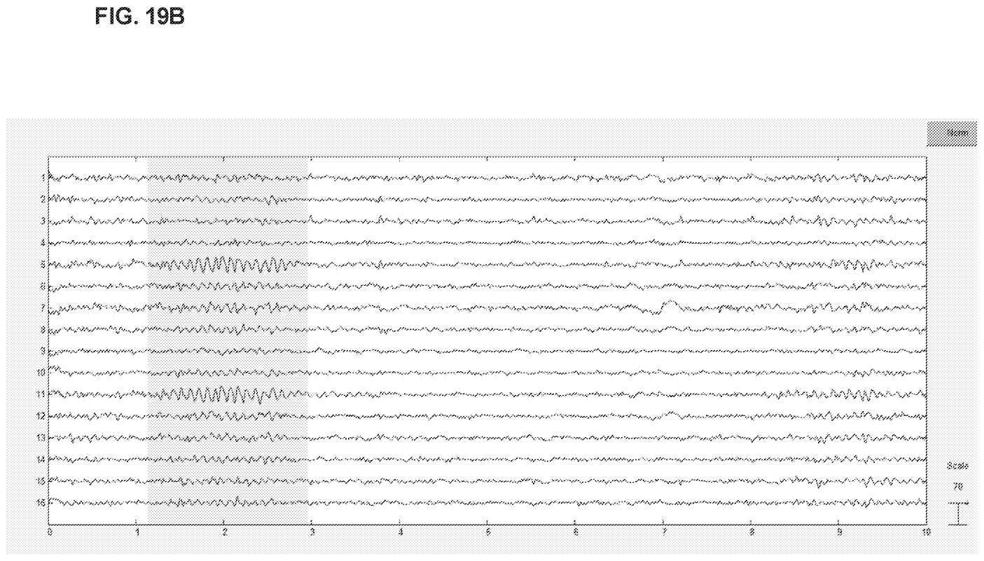

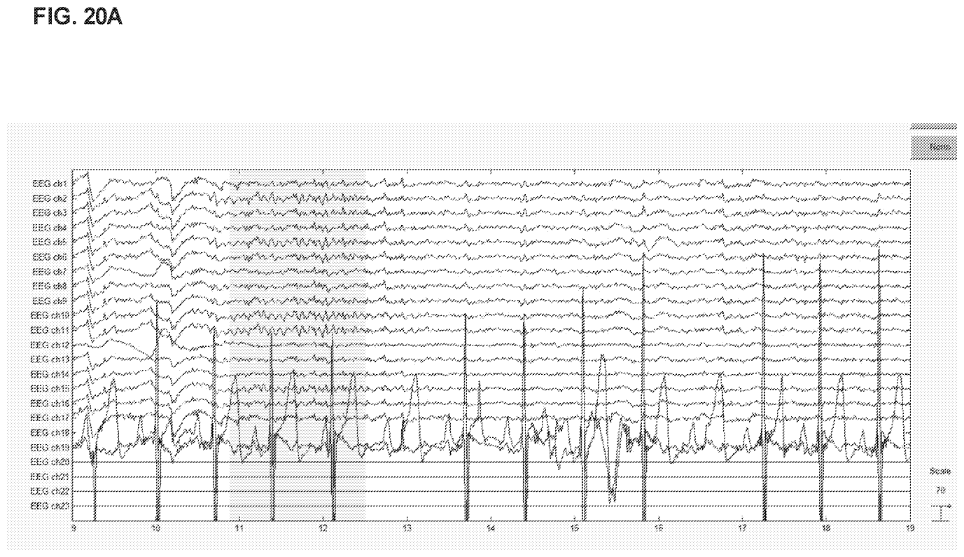

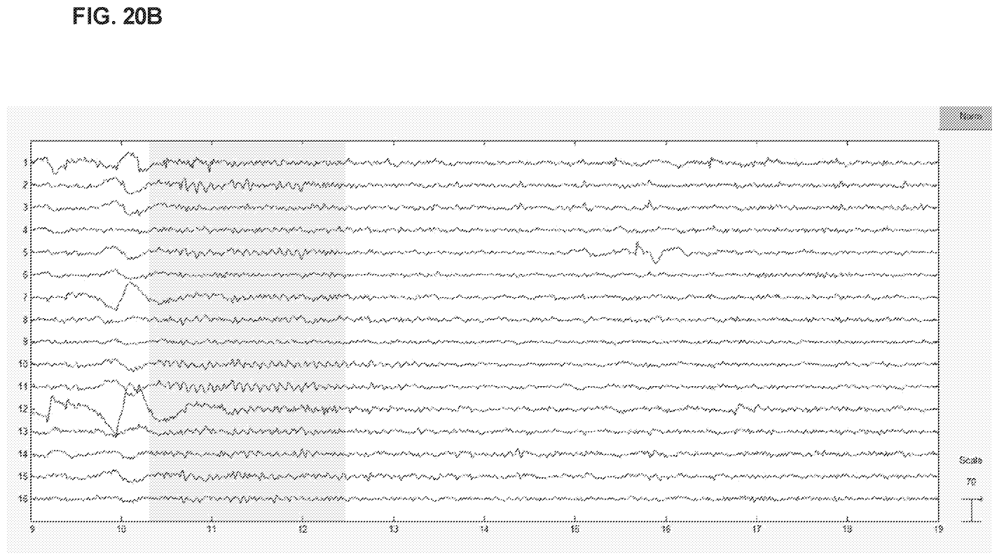

During the recording, the EEG signals were visualized in real time. The traces were processed and visualized using EEGLAB viewer (MATLAB) and two epochs containing posterior alpha rhythm (about 10 Hz) were detected (each epoch was recorded using different cap (Cap1 or Cap 2) and their timings were labeled. The results are presented in FIG. 19A (Cap1) and FIG. 20A (Cap2). The EEG data matrices were consequently processed using a corresponding algorithm. At the first stage, the data files containing EEG signals were loaded and thereafter, the EEG was re-referenced to the average of all electrodes on the cap. The results are shown in FIG. 19B (Cap 1) and FIG. 20B (for Cap 2).

At the next step, the timings of alpha rhythm epochs were recalculated from seconds to time-points (by multiplying seconds by sampling frequency).

Next, Fast Fourier Transform (FFT) was performed calculating frequency spectrogram absolute value (intensity) and phase. FFT was applied to 1 sec (500 timepoints) of the EEG after the beginning of alpha rhythm epoch, such that the frequency resolution (bin width) was 1 Hz.

The frequency bin of 10 Hz was selected for subsequent analysis.

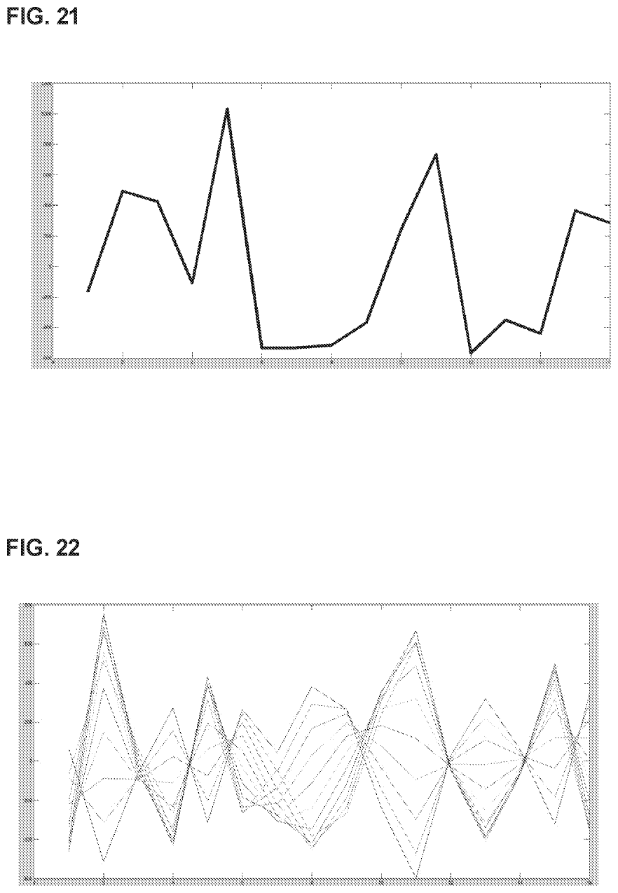

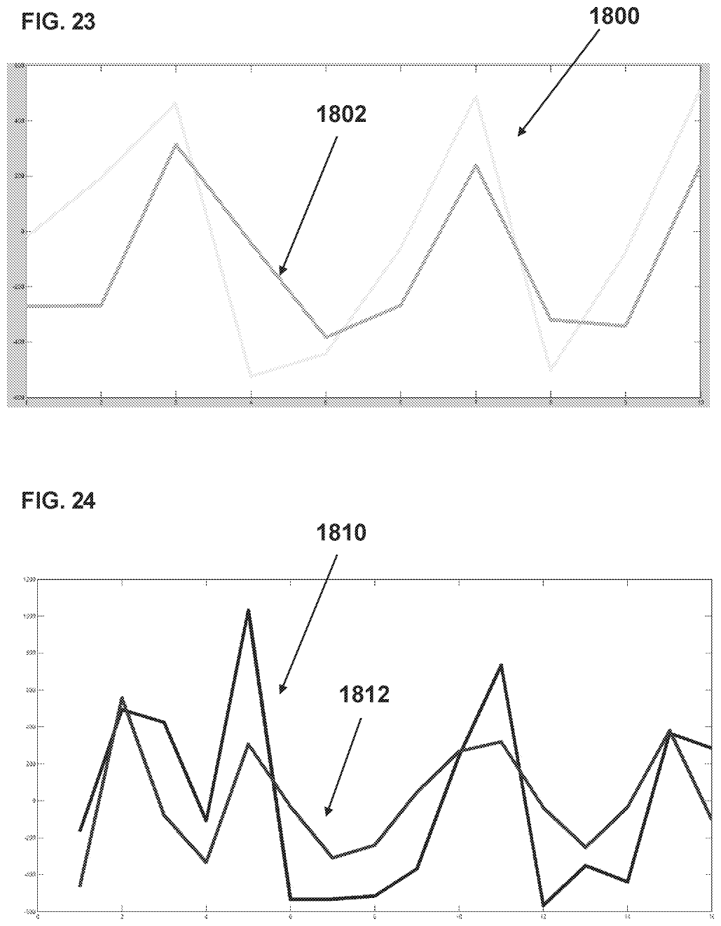

Since signals recorded at different times are not necessary keep the same phase, to superimpose such signals in frequency domine, one of the signals is rotated and the optimal phase is selected. This is achieved by two steps:
1. The real part of the 10 Hz-bin was calculated by multiplying the absolute value of 10-Hz bin to cosine of its phase. For cap 1 data the real part was calculated once. The cap 2 frequency data were rotated by multiplying the absolute value by cosine of changing phase from 0 to pi radians—with the steps of 0.1 pi radians. Thus, one real part of FFT was calculated for 10 Hz bin of cap 1 data (shown in FIG. 21) and 11 real parts—for cap 2 data (each with 0.1 pi radians rotated step) (shown in FIG. 22).
2. The values of interpolating points were calculated for electrodes of cap1 in longitudinal direction. Since the electrodes of cap2 were located between electrodes of cap1 in longitudinal direction, the field values in these interpolated points were expected to correspond approximately to values of cap2 channels. Accordingly, the real part of one of cap2 rotated FFT was selected by maximal correlation with cap1 interpolated points vector. (FIG. 23, in which line 1800 (Yellow line) represents the calculated field values (of 10-Hz bin) in the interpolated points between cap1 electrodes. And line 1802 (Green line) represents field values at cap2 electrodes corresponding to cap1 interpolated points. In this experiment the maximal correlation for 10 Hz-bin was 0.8862. (For example, for 2 Hz-bin, the maximal correlation was 0.4212 and for 5 Hz-bin it was 0.3991)

Since the optimal phase between signals of two caps was selected, the virtual dense array field could be reconstructed, providing that the signals measured by different arrays are the same power (or amplitude). Since the same brain generators at different type may exhibit different power, the signals measured at different time may be normalized according to their power. In this example, the field values were normalized using ratio between absolute values of 10 Hz-bin of alpha rhythm measured at different times.

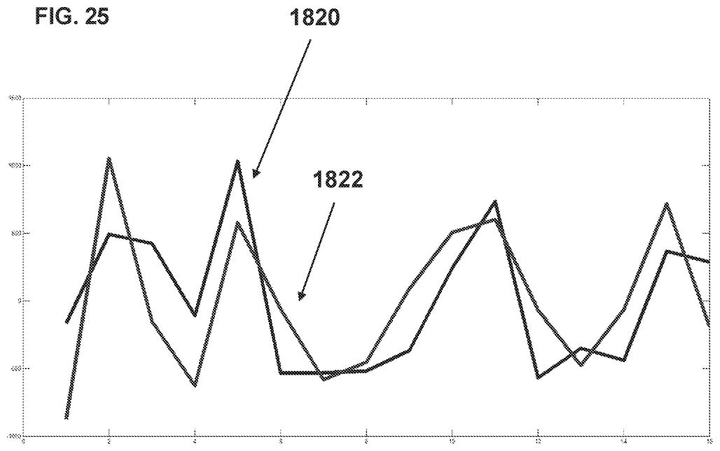

FIG. 24 demonstrates the combined field values of unnormalized signals (1810 line (Blue line)—cap 1. 1812 line (Red line)—cap 2. X-axis—channel numbers. Y-axis—real part of signal power in 10 Hz in 1 sec. FIG. 25 demonstrates the normalized signals measured by two different caps at different times, normalized by absolute value frequency signal power (in the 10 Hz-bin). In FIG. 25, 1820 line (Blue line—cap 1), line 1822 (Red line)—cap 2. X-axis—channel numbers. Y-axis—real part of signal power in 10 Hz in 1 sec (for cap 2—normalized according to cap 1) It is noted that most of the electrodes from the two different caps labeled by the same number were located in the neighboring position (FIGS. 18A-B), except of electrodes 2 and 4. This can explain the similarity of blue (1820) and red (1822) curves in FIG. 25.

### CONCLUSIONS

This experiment demonstrates that the same type of EEG signal recorded by different sparce EEG arrays at different times can be used for denser electric field reconstruction, constructing virtual EEG array.

In the experiment detailed above, posterior alpha rhythm was used since it is easier to generate such signals (by closing eyes), nevertheless different types of signals can be sampled and analyzed similarly, such as different types of epileptiform EEG signals. Similar results are obtained using a cap having alternating arrays, as disclosed and detailed herein.

What is claimed is:

1. A method for EEG monitoring of a patient, comprising:
positioning an array comprising a plurality of EEG electrodes on the patient's head;
recording, during a first time period, a first data set of EEG signals from a first set of spatial scalp locations;
manipulating said array to record, during a second time period, a second data set of EEG signals from a second set of spatial locations including at least some spatial scalp locations that are different from said first set of spatial scalp locations;
integrating said first and second data sets, thereby generating a combined data set with a higher spatial density; and
processing said combined data set to determine EEG events.

2. The method according to claim 1, wherein said first data set is recorded by a first set of electrodes of said array and said second data set is recorded by a second set of electrodes of said array.

3. The method according to claim 2, wherein said manipulating comprises moving said second set of electrodes from a position in which the electrodes are away from the scalp to a position in which the electrodes contact the scalp.

4. The method according to claim 1, wherein at least some locations of said first set of spatial scalp locations and of said second set of spatial scalp locations overlap.

5. The method according to claim 2, wherein said recording is carried out by placing said first set of electrodes or said second set of electrodes in pressurized contact against the scalp.

6. The method according to claim 2, wherein each of said first set of electrodes and said second set of electrodes include less than the total number of electrodes of said array.

7. The method according to claim 1, wherein a duration of each of said first and second time periods is shorter than 30 minutes.

8. The method according to claim 7, comprising repeating said recording from said first and second sets of spatial scalp locations in an alternating manner over a total duration of between 1-3 days or longer.

9. The method according to claim 1, wherein said processing comprises determining EEG events associated with epileptic seizures.

10. The method according to claim 1, wherein said integrating comprises representing data recorded by each electrode using a 4-dimensional vector which includes coordinates of the electrode position and a voltage amplitude recorded by the electrode.

11. The method according to claim 10, wherein said generating said combined data set comprises determining low spatial frequency components of EEG events from each of said first and second data sets and clustering said low spatial frequency components according to their temporo-spatial characteristics.

12. The method according to claim 11, comprising revealing high spatial frequency components of EEG events from said combined data set.

13. The method according to claim 1, further comprising assessing current electrode positions using photogrammetry methods.

14. A system for EEG monitoring of a patient, comprising:
a cap shaped and sized for fitting onto the head of the patient; the cap comprising:
a plurality of dry EEG electrodes including at least a first set of electrodes and a second set of electrodes;
an actuator configured to press said first set of electrodes against the patient's scalp to record EEG signals from a first set of spatial scalp locations, and then to press said second set of electrodes against the patient's scalp to record EEG signals from a second set of spatial scalp locations at least partially different than the first set of spatial scalp locations; and
a processor programmed to interpolate data recorded by said first set of electrodes and said second set of electrodes to determine EEG events.

15. The system according to claim 14, wherein said actuator is activated by a switch or timer.

16. The system according to claim 15, wherein when said actuator is activated, said actuator presses said first set of electrodes or said second set of electrodes against the scalp for a time period shorter than 30 minutes.

17. The system according to claim 16, wherein said electrodes are mounted onto inflatable tubes defined within said cap and wherein said actuator is configured to inflate said tubes to move said electrodes into contact with the scalp.

18. The system according to claim 17, wherein each electrode is operably attached to a spring, wherein deflation of a tube releases tension on said spring, causing said spring to bounce distally, thereby retracting the electrode away from the scalp.

19. The system according to claim 17, wherein said first set of electrodes is mounted onto a first tube or set of tubes, and wherein said second set of electrodes is mounted onto a second tube or set of tubes, said first and second tube or sets of tubes independently inflatable.

20. The system according to claim 14, wherein said electrodes are mounted or embedded within compression bands which extend at least across a long and transverse axis of the cap, the bands configured to be pulled on to push the electrodes against the scalp.

21. The system according to claim 14, wherein said cap comprises fixtures configured for attachment onto designated anatomical landmarks on the patient's head.

22. The system according to claim 21, wherein said landmarks comprise two or more of: the nasion, the inion and the preauricular point.

23. The system according to claim 14, wherein a size of said cap is adjustable via one or more pullable straps for obtaining a personal fit to the patient's scalp.

24. The system according to claim 14, comprising a user interface in communication with said processor, said user interface enabling self-activation by the patient or by a caregiver.

25. The system according to claim 14, wherein said processor is in communication with a remote server for transferring the recorded data and/or for comparing the recorded data onto data stored on the remote server.

26. The system according to claim 15, wherein said switch or timer are configured to activate said actuator to alternate between said first and second electrode sets throughout a total duration of between 1-5 days.

27. The system according to claim 15, wherein said cap is constructed such that each of said first and second sets of electrodes, when positioned in contact with the scalp, are distributed such between 2-8 electrodes are placed on each of the forelock, midscalp and crown regions of the scalp.

28. A system for EEG monitoring of a patient, comprising:
an array comprising a plurality of EEG electrodes;
an actuator configured for moving two or more of said plurality of electrodes together from a first position in which said electrodes are away from the patient's scalp to a second position in which said electrodes are in operable contact with patient's scalp.

29. The system according to claim 28, wherein said actuator is configured to maintain, at any given time during monitoring, at least some electrodes out of said plurality of electrodes at said first position away from the patient's scalp.

30. A system for EEG monitoring of a patient, comprising:
a cap shaped and sized for fitting onto the patient's scalp; the cap comprising:
a plurality of EEG electrodes embedded within it; the cap moveable from a first orientation relative to the scalp in which the plurality of electrodes are positioned to record EEG signals from a first set of spatial locations to a second orientation relative to the scalp in which the plurality of electrodes are positioned to record EEG signals from a second set of spatial locations; and
a processor programmed to integrate recordings of the first and second orientations to determine EEG events.

31. The system according to claim 30, wherein a total number of electrodes is smaller than the total number of spatial locations being recorded.

32. The system according to claim 30, wherein the total number of electrodes is half the number of spatial locations being recorded.

33. A method for long term EEG monitoring of a patient, comprising:
for a time period of between 1 hour-7 days, monitoring EEG of a patient by recording signals from different sets of spatial scalp locations; and
integrating data recorded from said different sets of spatial locations, wherein said integrating increases a spatial resolution of said data so that EEG events are detected as if said events were recorded from both of said sets of spatial locations.

34. The method according to claim 33, wherein said integrating increases said spatial resolution by between 20-200%.

35. An integrated EEG electrode and glue pad comprising:
a flexible glue pad onto which an EEG electrode sensor is centrally mounted, the flexible glue pad surrounded by frame which is rigid enough to prevent portions of the glue pad from sticking to each other.

36. A method for reducing noise from an EEG recording, comprising:
prior to recording EEG from a patient's scalp, short circuiting EEG electrodes by pairing the electrodes together;
assessing an artifact sensed by the short-circuited electrodes;
recording EEG from the patient's scalp using a different set of electrodes while maintaining said paired electrodes away from the scalp;
removing said artifact from the EEG data recorded by said different set of electrodes.

37. A method for long term EEG monitoring of a patient, comprising:
applying electrodes to the patient's scalp by positioning a first set of electrodes at a first distribution along a scalp region of interest; and a second set of electrodes at a second distribution along the other scalp regions;
wherein electrode density of said first distribution is at least 60% higher than electrode density of said second distribution.
